(19) United States
(12) Patent Application Publication
(10) Pub. No.: US 2025/0085220 A1
(43) Pub. Date: Mar. 13, 2025

(54) METHOD FOR MONITORING A CONTROL PARAMETER ON A SUBSTANTIALLY PLASTIC MATERIAL, RELATING TO APPARATUS AND PYROLYSIS PROCESS WHICH USES THIS METHOD

(71) Applicant: Versalis S.p.A., San Donato Milanese, Milano (IT)

(72) Inventors: Riccardo FELISARI, San Donato Milanese, Milano (IT); Armando GALEOTTI, San Donato Milanese, Milano (IT); Mirco NODARI, San Donato Milanese, Milano (IT); Francesco BONACINI, San Donato Milanese, Milano (IT); Antonio PONTICIELLO, San Donato Milanese, Milano (IT)

(73) Assignee: Versalis S.p.A., San Donato Milanese, Milano (IT)

(21) Appl. No.: 18/725,831

(22) PCT Filed: Dec. 27, 2022
(86) PCT No.: PCT/IB2022/062801

§ 371 Date: Jul. 1, 2024

(30) Foreign Application Priority Data
Dec. 30, 2021 (IT) 102021000033059

(51) Int. Cl. G01N 21/3577; G01N 1/44; G01N 33/44

(52) U.S. Cl. CPC G01N 21/3577; G01N 1/44; G01N 33/442

(57) ABSTRACT

The present invention is related to the treatment of plastic material to be destined for chemical recycling processes for the valorisation of substantially plastic material otherwise destined for disposal.

In particular, the invention concerns a process for the pyrolysis of substantially plastic material to obtain at least liquid hydrocarbons at 25° C. comprising the following steps:
a) feeding to a pyrolysis reactor a substantially plastic material at least partially in the molten state;
b) subjecting said substantially plastic material fed to a pyrolysis reactor to at least an analytical measurement “Ax” with in-line mode;
c) determining the value of at least one “Px” property of said substantially plastic material by means of said at least one analytical measure “Ax”;
d) installing at least one “Ox” parameter of the pyrolysis process based on the value of this at least one “Px” property;
   characterised by the fact that at least one analytical measurement “Ax” provides or consists of the measurement of the spectrum in reflection of the light of the substantially plastic material.

The present invention is relative to the treatment of plastic material to be destined for chemical recycling processes for the valorisation of substantially plastic material otherwise destined for disposal.

In particular, the present invention concerns a method to monitor a parameter of control on a substantially plastic material, the relative apparatus and a pyrolysis process of substantially plastic material which uses said method.

Advantageously, the present invention can be applied to treat substantially pre-processed plastic material in a sorting plant, in which some types of plastic material are identified and separated as individual polymers.

In this way, the fractions recoverable as a single typology of the polymer can be reused as such (through so-called “mechanical” or “physical” recycling), and only the part not recoverable as a single polymer is subjected to pyrolysis. Following pyrolysis, hydrocarbon products are produced which are subjected to further treatments, such as steam cracking, generate monomers which can then be polymerised forming new plastic. This allows closing the cycle of the plastic, that is realising the so-called “closed loop recycling”.

The recovery of said substantially plastic material, especially the residue after sorting, is particularly difficult since having already selected what can be recovered as a single polymer (for example, polyethylene and polyethylene terephthalate), the residue becomes extremely varied and also includes non-plastic materials which are difficult to recover.

Through the method, apparatus and process described in the present invention, which teach an innovative way of analysing said substantially plastic material, which is in itself very heterogeneous, and of performing pyrolysis according to the results of the analysis carried out, it is therefore possible to close the plastic cycle (thus also recovering said residue by “chemical” method) efficiently.

### KNOWN ART

There are many articles and patent applications on plastic material pyrolysis processes, but only a very small number also disclose measurement systems on the raw material treated. EP3405297 discloses apparatus a method and for the classification of tyres on the basis of silicon content (silicon lesser or greater than 15% by weight). Said silicon content is measured with a sensor based on electrical resistivity, X-ray fluorescence, absorption in the near infrared (NIR) and laser-induced plasma spectroscopy (LIPS).

A method is also disclosed for carrying out the selective pyrolysis of tyres with high silicon content, in order to obtain a char residue with a high silica content.

WO2012172527 discloses a method for the continuous recycling of plastic and transformation into liquid fuels, comprising feeding the plastic into a rotating tank, which facilitates the transport towards the end of the products obtained by angular motion, by continuously removing non-gaseous products. One method described comprises the preventive separation of unwanted plastic materials, i.e. which include “heteroatoms”. According to a further method, said separation is carried out by means of an NIR system, capable of identifying the different plastic materials which include heteroatoms based on NIR spectra, in order to separate said fractions with the help of an ejector gas.

In the known art, the NIR spectrum is analysed by a spectrometer in transmission or transmittance mode.

In transmission spectroscopy, the radiation analysed by the spectrometer is the fraction of the incident radiation which passes through the sample, i.e. the fraction which is neither absorbed or reflected by this.

In transflection spectroscopy, the radiation analysed by the spectrometer is the fraction of the incident radiation which, after passing through the sample, is reflected by a special reflecting screen placed in the measuring cavity along the path of the radiation, beyond the sample; the radiation reflected from the screen passes through the sample a second time, before reaching the spectrometer.

There are finally several patents and scientific articles which relate to the pyrolysis process carried out at pressures different from atmospheric pressure

EP2348254 describes a “zero emissions” pyrolysis process conducted at 10-15 bar and, simultaneously, feeding a flow of pure oxygen.

ES2389799 discloses a process for the production of diesel fuel (C13-C40) which foresees two stages, under pressure (1-15 bar). The first stage is thermal, while the second is catalytic, and in the presence of hydrogen. The input material is preferably of polyolefin origin. It may contain polystyrene, but preferably the content of other plastics such as PVC and PET is lower than 10%.

There are also multiple inventions which disclose pyrolysis carried out at reduced (sub-atmospheric) pressure, such as WO2013187788, WO0231082 and EP2184334.

From the analysis of the known art it is clear that the measurement of properties on the plastic material which is then fed to a plant of pyrolysis is carried out for the sole purpose of selecting the power supply to the pyrolysis reactor, or to classify the product obtained. In particular, the analysis carried out is not used for the control of the pyrolysis process. There are also several pyrolytic processes carried out at pressures lower or higher than atmospheric pressure, however there is no generalised teaching which allows us to establish whether it is advantageous to manage a pyrolytic process of complex polymeric mixtures at atmospheric pressure, at sub-atmospheric (reduced) pressure or in overpressure (i.e. at a pressure higher than atmospheric pressure). The information available is often very varied, sometimes contradictory and provides completely different indications. In addition, none of the documents identified provide information on how to set process parameters such as the operating pressure of the pyrolytic system according to the quality of the polymer fed. In no case does the fed material have an inconsistent composition, and in any case it is not known how to manage the change in composition and the effect on the process and on the product obtained.

The substantially plastic material remaining after the process of selection and extraction of single polymers is instead by its nature of very variable composition and composed of multiple types of plastic materials, as well as non-plastic materials.

Furthermore, the pyrolysis processes need the plastic fed to them to have been previously selected in order to reduce the quantity of difficultly treatable plastics (such as PVC, PET, cellulose, polystyrene) and non-plastic materials, favouring instead polyolefins (especially polyethylene and polypropylene). However, most of the polyolefins contained in the substantially plastic material are generally separated in said selection processes, and then recycled as such without having to carry out a pyrolysis. There is therefore an interest in treating in pyrolysis above all the residual fraction after selection, which contains, in addition to polyolefins, significant quantities of other plastics and smaller quantities of non-plastic materials.

A further problem associated with these spectroscopic modes is linked to the fact that the substantially plastic material is often of variable composition and also includes non-plastic materials, such as carbon black and graphite, which even in small quantities obscure the transmitted radiation, thus making the reading of the spectrum problematic. Furthermore, the substantially plastic material can be partially degraded and therefore partially opaque, in particular in the presence of high amount of oxygen. Finally, polymer compounds are typically incompatible. Therefore, the polymeric melt thus produced is typically opaque and inhomogeneous.

In addition, the evaluation of the composition of a substantially plastic material as such (i.e. on the particulate as received from the supplier) is imprecise because it is not homogeneous (generally each individual particulate being of a different material). In addition, the analysis also depends on uncontrollable variables such as the size and aspect ratio of the particulate matter. The evaluation of the composition on the substantially plastic material already in the melted and mixed state, although it can solve these criticalities, is generally difficult to achieve with the spectroscopic techniques known in the art for the reasons set out above.

Finally, a method would be desirable that can determine at least the characteristics of the composition which have the greatest influence on the pyrolysis process in a short time, so that there is low latency between the feeding of the substantially plastic material into the pyrolysis reactor and the modification of the process such as temperature, pressure or residence time. In fact, typically the residence time of said material in the molten state before it is sent to the pyrolysis reactor is very short.

For the reasons indicated above, there is therefore a need to identify a process and related apparatus which can overcome the limitations of the known art.

### SUMMARY OF THE INVENTION

The Applicant has surprisingly found a process for the pyrolysis of substantially plastic material to obtain at least hydrocarbons that are in liquid phase at 25° C. by subjecting a substantially plastic material to a specific pyrolytic process, even of non-constant composition, optionally also comprising high quantities of components normally considered undesirable.

This process includes the steps of:
a) feeding a substantially plastic material at least partially in the molten state to a pyrolysis reactor;
b) subjecting said substantially plastic material fed to a pyrolysis reactor to at least one analytical measurement “Ax” with in-line mode;
c) determining the value of at least one property “Px” of said substantially plastic material by means of said at least one analytical measurement “Ax”;
d) set at least one parameter “Ox” of the pyrolysis process based on the value of said at least one property “Px”;
characterised by the fact that said analytical measurement “Ax” provides for or consists of the measurement of the light reflection spectrum of the substantially plastic material analysed.

In one embodiment, this process also includes also the step of:
e) iteratively repeating steps b), c) and d)

One first advantage of the process disclosed in the present invention is the ability of the process to treat substantially plastic material of even highly variable composition, without the need to stop the process when the type of substantially plastic material fed is changed.

One further advantage of the process disclosed in the present invention is that, integrated in a pre-selection process, it allows the recycling of plastic an indefinite number of times (“closed loop recycling”); that is, such that the material can be used and then regenerated several times without losing its properties during the recycling process.

One further advantage of the process disclosed in the present invention is the ability of the process to treat substantially plastic material comprising vinyl polymers (polyethylene and polypropylene), polyvinylaromatic polymers, such as polystyrene (PS) and its alloys, non-vinyl polymers, such as for example polyethylene terephthalate (PET), and polymers rich in oxygen, such as for example cellulose and PET itself, without any process problems such as fouling or occlusions, and with high quality of said hydrocarbons that are in liquid phase at 25° C.

One further advantage of the process disclosed in the present invention is the ability of the process to treat substantially plastic material also comprising high quantities of components normally considered undesirable such as paper and cardboard (cellulose) and chlorinated or brominated compounds, such as polyvinyl chloride (PVC) and polymers containing halogenated flame retardants.

One further advantage of the process disclosed in the present invention is the ability of the process to treat substantially plastic material of non-constant composition, without fouling and occlusions occurring.

One further advantage of the process disclosed in the present invention is the ability of the process to treat substantially plastic material consisting of the residue that it has not been possible to separate and recycle in the selection processes which are generally applied to plastic waste.

One further advantage of the process disclosed in the present invention is the ability of the process to produce a high quality pyrolysis oil in terms of the obtained composition, even when the substantially plastic material treated is of non-constant composition, maintaining a substantial high quality of the produced hydrocarbons that are in liquid phase at 25° C.

One advantage of the apparatus disclosed in the present invention is the ability to operate continuously and for long periods of time without the need for interruptions for maintenance and cleaning.

A further advantage of the apparatus disclosed in the present invention is that the encumbrance of the probe and of the detection system inside the cavity is minimised, in particular in the way in which these elements are arranged in a single integrated device. This reduced encumbrance reduces the pressure losses suffered by the substantially plastic material in the measuring cavity and reduces fouling in the same.

A further advantage of the process and apparatus disclosed in the present invention is that the measurement is performed in-line (also known as in-situ).

In fact, on-line measurement systems are known in the art in which the sample to be analysed is taken from the main process flow, continuously or discontinuously. Said transfer, from the process line to the measuring instrument, takes place through a transfer line. The problem of fouling of the sampling windows remains, as the polymerisation mixture must in any case pass through a measuring cell (located outside the lines dedicated to the pyrolysis process and connected to it through the transfer line) with problems which are completely similar to those of the measuring cavities of the immersion probes. Furthermore, in many cases, during the transfer of the sample of substantially plastic material to be analysed from the reactor to the measuring instrument, a structural alteration of the sample itself may occur, which can also significantly affect the outcome of the determination of the “Px” property evaluated (for example, unmixing, degradation, gas development may occur). Finally, the on-line measurements suffer from a delay in the measurement, often difficult to quantify, as the material taken from the main flow at a given moment will be measured by the measuring instrument placed downstream of said transfer line only after a time equal to the residence time of the material in said transfer line.

The method of measuring the property “Px” proposed in the present invention is instead in-line, i.e. the measurement is carried out on the same flow of substantially plastic material that is fed to the pyrolysis reactor, or without the need for transfer lines for the material to be analysed.

A further advantage of the process disclosed in the present invention is that the latency time between the analytical measurement “Ax” and the setting of the process parameter “Ox” is not more than one hour, preferably not more than 10 minutes, even more preferably between 1 and 60 seconds. Considering that the time necessary for the substantially plastic material thus analysed to enter the reactor is typically greater than 1 second, ensuring said latency time implies that the adjustment of the “Ox” process parameter can be carried out, if necessary, in a time not exceeding 10 minutes, preferably within 59 seconds.

The critical issues illustrated above regarding the state of the art have so far prevented an adequate exploitation of the potential of spectroscopy in monitoring the properties of the substantially plastic material in a pyrolysis plant, in particular where information is to be obtained from these with a low latency time, or with a reduced time delay between the determination of the property in question and the processing of the same sample in pyrolysis.

Therefore, in current practice the pyrolysis is normally carried out under constant conditions, and therefore independently of the substantially plastic material fed. Sometimes, empirically, experience has been gained regarding the optimal processing conditions as a function of the type of substantially plastic material. For example, it is assumed that the substantially plastic material coming from a given supplier is of constant composition, so that over time empirical process recipes (temperature, residence times, flow rate, etc.) optimised for the substantially plastic material are originating. from a specific supplier.

Probably, it would also be possible to carry out off-line measurements, taking a sample of substantially plastic material in spot and analysing it with a spectrometer in the laboratory, and adapting said process recipe according to the accumulated empirical experience. However, this method is decidedly disadvantageous, as inevitably the processing times of the analysis by a laboratory cannot be too short, and therefore to take advantage of the information obtained in the laboratory it is necessary that the pyrolysis process can only be started after obtaining them.

Furthermore, in this mode the substantially plastic material analysed can only be a sample. Considering the high variability and uncontrollability of the composition of substantially plastic material to be processed, the sample analysis may not be representative of the substantially plastic material actually processed; therefore the process conditions determined may be erroneous.

The object of the present invention is therefore to overcome or at least mitigate the drawbacks of the state of the art highlighted above, in particular by providing a process for the pyrolysis of substantially plastic material which allows to maintain a high qualitative constancy of said hydrocarbons that are in liquid phase at 25° C. obtained by said pyrolysis process, even when the substantially plastic material fed to the pyrolysis process is not constant.

The advantages obtained and disclosed herein, which make use of reflectance spectroscopy in accordance with the present invention, are to be considered even more surprising if one considers that the intensity of the radiation reflected by the polymerisation mixture that can be collected by a probe (and subsequently processed by the detection system) is decidedly lower than that which can be collected with measurement probes in transmittance or transflectance.

The present invention also relates to a mixture which includes hydrocarbons in quantities greater than 90% by weight and tetrahydrofuran in quantities between 0.01% and 0.25% by weight, with respect to the total weight of the mixture and the use of said mixture to feed a cracking plant.

A further aspect of the present invention is the method for determining the value of said at least one property “Px” of said substantially plastic material, which can be used to carry out steps b) and c) of the above pyrolysis process. Said method for determining the value of at least one property “Px” of said substantially plastic material comprises the steps of:
(i) calculating, for each of said at least one property “Px”, a calibration curve “Cx” which, for sample materials having a known value of the property “Px”, correlates the spectrum in reflection with said property “Px”;
(ii) carrying out at least one measurement of the reflection spectrum of said substantially plastic material;
(iii) determining the value of said at least one property “Px” of said substantially plastic material starting from said at least one calibration curve “Cx”;
   and that it is characterised in that said substantially plastic material, in the conditions of measurement of spectrum in reflection, is in the molten state for at least 60% by weight, preferably between 70% and 99% by weight, even more preferably between 1 80% and 94% by weight of the material, with respect to the overall weight of the substantially plastic material.

Preferably, said substantially plastic material under the measurement conditions has a temperature comprised between 14° and 300° C., even more preferably between 16° and 260° C.

Preferably, said substantially plastic material under the measurement conditions has a pressure of not less than 2 bara, even more preferably between 3 and 300 bara.

According to a preferred method, called “multiple correlation”, there are at least two at least one “Px” property. According to an even more preferred modality, called “double correlation”, there are two at least one “Px” property.

According to this preferred modality with double correlation, preferably the “Px” properties are the hydrogen index on carbon (H/C index) and the carbon index.

Preferably, the reflection spectrum of said substantially plastic material acquired in step ii) is included in the range between 4000 and 12000 cm⁻¹, even more preferably it is included in the range between 4500 and 10000 cm⁻¹ and even more preferably it is included in the range between 5000 and 9000 cm⁻¹.

Preferably, the “Cx” calibration curve is obtained by applying a multivariate regression method. Preferably, said multivariate regression method is multiple linear regression (MLR), regression by principal component analysis (PCR) or regression by partial least squares (PLS). Even more preferably, multivariate regression is partial least squares (PLS) regression.

Finally, the present invention also concerns the apparatus for determining the value of at least one property “Px” of a substantially plastic material at least in part in the molten state, which includes:
1) a cavity in which said substantially plastic material can slide, provided with an inlet and an outlet for said substantially plastic material at least in part in the molten state;
2) at least one probe capable of emitting light in said cavity;
3) at least one detection system optically coupled with said probe for detecting reflected light and the determination of an analytical measure “Ax”;
4) at least one sensor for reading the temperature connected to said cavity;
5) optionally, a system for determining said at least one property “Px” of said substantially plastic material at least in part in the molten state by correlating said at least one analytical measurement “Ax” with a calibration curve “Cx” obtained from sample materials having a value known of the property “Px”,
wherein said cavity is able to withstand a pressure of at least 50 bar, preferably from 80 to 300 bar.

Preferably, said sensor for reading the temperature is positioned so that the minimum distance between the temperature reading area and the area where the reflection spectrum is measured is not greater than 100 mm, preferably not greater than 30 mm, even more preferably not more than 3 mm.

The temperature reading zone is the surface where the temperature sensor detects the temperature. For devices such as thermocouples and thermistors, where the detected temperature is that at the end of the same, said area therefore corresponds to the surface of said extremity.

Preferably, said at least one detection system optically coupled with said probe is positioned so that the minimum distance between the emission zone of the probe and the detection zone of said emission is not greater than 50 mm, preferably not greater than 15 mm, again more preferably not greater than 3 mm.

Preferably, said probe is capable of emitting light having a wavenumber included at least in the range between 4000 and 12000 cm⁻¹, even more preferably it is included in the range between 4500 and 10000 cm⁻¹ and even more preferably it is included in the range ‘range between 5000 and 9000 cm⁻¹. Preferably, the “Px” property is the “hydrogen on carbon” index (H/C index) and/or the carbon index.

According to one preferred method, said cavity is constituted by at least one part of the cylinder of an extruder. According to this preferred method, the extruder can be used to melt at least part of the material and optionally remove the gaseous compounds.

According to one alternative method, said cavity is the cavity of a conveyor screw connected to the inlet of substantially plastic material in the pyrolysis reactor.

According to one alternative method, said cavity is a section of piping connected to the inlet of substantially plastic material in the pyrolysis reactor.

According to one alternative method, said cavity is inside the inlet of the pyrolysis reactor.

According to one alternative method, said apparatus for determining the value of at least one property “Px” of a substantially plastic material also comprises a window made of a substantially transparent material in the wavenumber range of said detection system optically coupled with said probe, interposed between said probe and said cavity, and/or between said optically coupled detection system and said cavity.

In this alternative modality, the window allows the physical separation between the substantially plastic material to be analysed and the probe and/or detection system. In this way it is therefore possible to use probes and/or detection systems which do not necessarily have to guarantee sealing and resistance to the typical high pressures and temperatures of a substantially plastic material in the molten or semi-molten state.

### Definitions

In the description of the present invention, unless otherwise specified, the values of the ranges (for example, ranges of pressure, temperature, quantity, etc.) are to be considered including extremes.

In the description of the present invention, unless otherwise specified, the percentages are to be understood by weight (i.e. by mass). The symbol “%” means percent, always by weight (mass).

In the description of the present invention, the term “comprise” also includes as a particular limiting case its meaning as “consist of” or “consist in”.

In the description of the present invention, the term “essentially consists of” or “essentially consists in” means that the composition or formulation (A) necessarily includes the listed ingredients and (B) is open to unlisted ingredients which do not materially affect the basic and new properties of the composition.

In the description of the present invention, a material is in the “molten state” at a given temperature if it is not in the solid state and has a melt flow rate (MFR), measured in accordance with ISO 1133-1:2011, under a weight of 10 kg and at said temperature, higher than 2 grams in ten minutes.

In the description of the present invention, the “melted state” therefore also includes the liquid state.

In the description of the present invention, the term “meltable substance” means that it is possible to bring said substance to conditions whereby said substance is in the molten state at a temperature below 300° C. For example, meltable substances are substances which are already liquid at room temperature, such as water, or solid substances at room temperature like most thermoplastic polymers. For example, inorganic salts and inorganic carbon compounds such as carbon black, coke, graphite, or polymers such as polyether ether ketone (PEEK) are not meltable substances.

In the description of the present invention, with substantially plastic material “at least partially in the molten state” it is meant that the material is in the molten state for at least 60% by weight, preferably between 70% and 99% by weight, even more preferably between 80% and 94% by weight of the material, with respect to the overall weight of the substantially plastic material.

In the description of the present invention, by “sample spectrum” is meant a spectrum obtained on a sample material.

In the description of the present invention, unless otherwise specified, to maintain a certain parameter (for example the pressure) within an indicated range, it is meant that one actively operates so that this parameter falls within the range, for example by checking that the value measured falls within the indicated range, and/or regulation of the parameter by means of a feedback regulation system in which a value of this parameter is set within the indicated range.

Preferably, keeping a certain parameter (for example the pressure) at the set value or within an indicated range indicates that this parameter is set in a feedback control system at a value within the indicated range, so as to bring the parameter at the set value or within the indicated range.

In the description of the present invention, unless otherwise specified, by hydrocarbons that are in the liquid phase at 25° C. we mean the hydrocarbon mixtures which are in the liquid phase at 25° C. and at atmospheric pressure.

In the description of the present invention, by pyrolysis oil is meant the product of pyrolysis which is in the liquid state at 25° C. and at atmospheric pressure.

In the description of the present invention, by “pyrolysis vapours” we mean the product which is generated during the pyrolysis process which is in the gaseous state in the pyrolysis reactor, i.e. which is in the gaseous state under the conditions of temperature, pressure and composition of the pyrolysis.

In the description of the present invention, “pyrolysis residue” means the product which is in the liquid, solid or liquid state and solid in the pyrolysis reactor, that is, which is in the liquid and/or solid state under the conditions of temperature, pressure and composition of pyrolysis.

In the description of the present invention, unless otherwise specified, for a value of a parameter equal to at most a certain value X it is meant that the parameter is equal to X or less than X; and for a value of a parameter equal to at least a certain value X it is meant that the parameter is equal to X or greater than X.

In the description of the present invention, unless otherwise specified, by yield in the production of a product is meant the percentage by weight of that product with respect to the total of products made.

Unless otherwise specified, in this document “part” and “parts” mean respectively part by weight and parts by weight. Weight means mass, i.e. kg in SI units.

### DESCRIPTION OF THE FIGURES

FIG. 1 shows a cavity in which substantially plastic material flows, including an integrated device which comprises a probe capable of emitting light, a detection system optically coupled with said probe, and a thermocouple for detecting the temperature of the substantially plastic material illuminated by said light;

FIG. 2 shows an embodiment example of said integrated withdrawable device;

FIG. 3 shows the predictive capacity of the calibration curve “Cx” corresponding to the property “Px” carbon index and H/C index (H/C index), for the PLS (Partial Least Squares) regression, in which:
in the abscissa is shown the number of main components used (NC)
in the ordinate is shown the mean square error in cross-validation (RMSECV, root mean square error in cross validation); the unit of measurement is that corresponding to the property “Px” (the H/C index in the case of polythene/polypropylene is 100);

FIG. 4 shows the predictive capacity of the “Cx” calibration curve corresponding to the “Px” H/C index property (H/C index), for the 51 sample materials examined; There are almost overlapping points so that in the figure there seem to be fewer than 51 points, in which the “true” H/C value is shown in the abscissa, that is the one determined by the primary analysis, and in the ordinate the H/C value calculated with the corresponding calibration curve “Cx”;

FIG. 5 shows the predictive capacity of the “Cx” calibration curve corresponding to the “Px” carbon index property, for the 51 sample materials examined;

FIG. 6 shows some absorption spectra made on the sample material M48 at different temperatures of the sample material, in which the wave number is reported in the abscissa (in cm 1) while the absorbance is reported in the ordinate;

FIG. 7 schematically shows an apparatus for the pyrolysis of substantially plastic material to obtain at least hydrocarbons that are in the liquid phase at 25° C. according to the invention;

FIG. 8 shows an embodiment of the split range control mode according to the present invention;

FIG. 9 shows a temperature vs. time relative to the two temperature profiles T1 and T2 used in the pyrolysis process of the examples.

### DETAILED DESCRIPTION OF THE INVENTION

The present invention primarily relates to a process for the pyrolysis of substantially plastic material to obtain at least hydrocarbons that are in the liquid phase at 25° C. comprising the steps of:
a) feeding a substantially plastic material at least partially in the molten state to a pyrolysis reactor;
b) subjecting said substantially plastic material fed to a pyrolysis reactor to at least one analytical measurement “Ax” with in-line mode;
c) determining the value of at least one property “Px” of said substantially plastic material by means of said at least one analytical measurement “Ax”;
d) setting at least one “Ox” parameter of the pyrolysis process based on the value of said at least one “Px” property; characterised by the fact that said analytical measurement “Ax” provides for or consists of the measurement of the spectrum in reflection of the light of the substantially plastic material.

According to the present invention, the sample of substantially plastic material is irradiated with a light by means of a suitable light source and a reflection spectrum of said substantially plastic material subject to said light source is then acquired by means of an instrument called a spectrometer.

Preferably, said spectrometer is a spectrophotometer, that it is equipped with a system for the quantitative measurement of light intensity.

There are no particular limitations on the type of spectrometer. For example, spectrometers with prism or grating monochromator, or Fourier transform spectrometers, known as FTIR, can be used.

Monochromatic spectrometers can advantageously comprise a series of photodiodes (“photo diode array” or PDA, otherwise also known as “diode array”). Said spectrometers are also known with the term “DAS” (diode array spectrometer) or PDAS (photo diode array spectrometer). Alternatively, sensors and corresponding CCD (charged-coupled device) spectrometers can also be used.

The sample of substantially plastic material to be measured is irradiated by a broad spectrum light, that is, which includes all the frequencies included in the range of wave numbers being measured.

Said spectrum is preferably in the visible, i.e. between 12000 and 25000 cm⁻¹, in the near infrared (NIR), i.e. between 4000 and 12000 cm⁻¹ and/or in the medium infrared (MIR), i.e. between 400 and 4000 cm⁻¹. More preferably, said spectrum is between 4000 and 12000 cm⁻¹, even more preferably it is in the range between 4500 and 10000 cm⁻¹ and even more preferably it is in the range between 5000 and 9000 cm⁻¹.

The light reflected by the substantially plastic material sample is collected by a detection system optically coupled with said probe which emits said light source.

By system optically coupled with said probe emitting said light source it is meant that the detection system is positioned in such a way as to be able to receive the light emitted by said source and reflected by the sample.

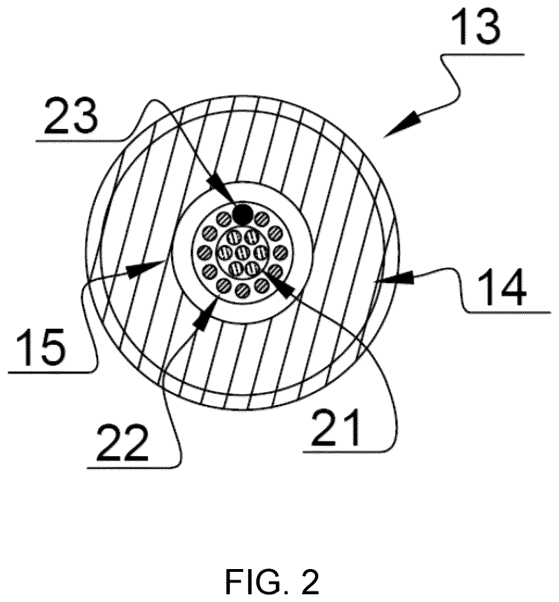

For example, said probe and said detection system can be constituted by optical fibres, joined in a single integrated device, as shown in FIG. 2.

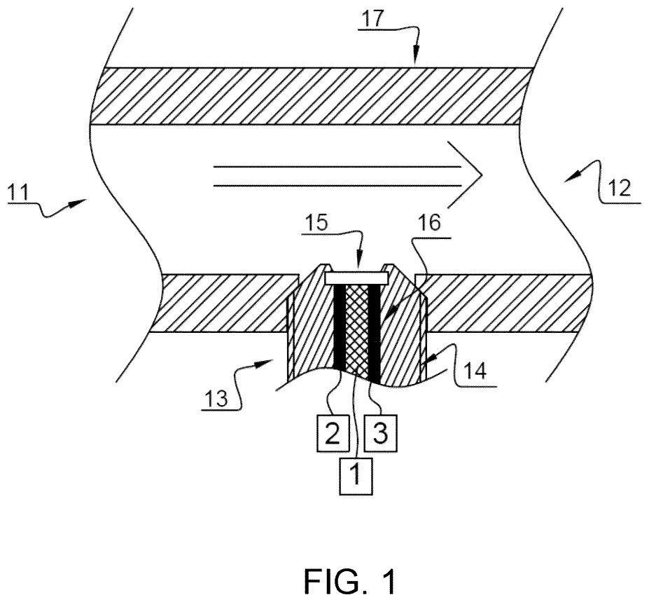

With reference to FIG. 2, an embodiment is reported in which the following are visible:
an integrated extractable device (13);
a body (14) of the device (13);
a window (15);
optical fibres (21) which constitute the probe capable of emitting light, arranged centrally and connected to the light source (1) (not shown in FIG. 2 but only in FIG. 1);
optical fibres (22) which constitute the detection system optically coupled with said probe (21), arranged peripherally but in proximity to said probe capable of emitting light (21), and connected to the spectrum analyser (2) (not shown in FIG. 2 but only in FIG. 1);
a thermocouple (23), placed peripherally but in proximity to said probe capable of emitting light (21), and connected to the temperature reader (3) (not shown in FIG. 2 but only in FIG. 1).

Since the fibres of the probe and of the detection system are close and aligned, and the substantially plastic material being in contact with the window, it is ensured that the light emitted illuminates said substantially plastic material and that the reflected light reaches said detection system.

According to an alternative modality of the present invention, the optical fibres of the probe capable of emitting light are located externally with respect to the optical fibres of the detection system optically coupled with said probe. Still referring to FIG. 2, this means that, in this mode, the optical fibres (22) are those of the probe capable of emitting light while the optical fibres (21) are those of the detection system probe optically coupled with said probe.

The sampling window (15) is made of a material which is substantially transparent to light radiation in the wave number range of the spectrum analysed. For example, it can be made of quartz, sapphire, potassium bromide or zinc selenide.

FIG. 1 instead illustrates a cavity in which substantially plastic material flows, including an integrated device that includes a probe capable of emitting light, a detection system optically coupled with said probe, and a thermocouple for detecting the temperature of the substantially plastic material illuminated by said light, in which are visible:
a hollow body (17) inside which said substantially plastic material slides;
an inlet (11) and an outlet (12) of said substantially plastic material, with the indication of the sliding direction of the substantially plastic material by means of an arrow;
an integrated withdrawable device (13) which includes:
   a body (14) solidly connected to said hollow body (17) by threading;
   a window (15) made of substantially transparent material in the wavenumber range of the spectrum;
   a central cavity (16) in which the optical fibres are arranged for the emission and detection of light and the thermocouple that reads the temperature of the substantially plastic material illuminated;
a connection of said integrated extractable device (13) with the light source (1), the spectrum analyser (2) and the temperature reader (3).

If an integrated device is used, which therefore comprises both said probe for the emission of light and said optically coupled detection system, it is advantageous for said device to be positioned substantially perpendicular to the direction of motion of the substantially plastic material, that is, so that the emitting and receiving optical fibres are substantially perpendicular to said direction of motion (as shown in FIG. 1).

Again in the case in which an integrated device is used, it is advantageous that said device protrudes into said cavity where the substantially plastic material flows for no more than 3 cm, preferably for no more than 1 cm, even more preferably for no more than 0.5 cm

However, it is not excluded that said integrated device can be inserted in said cavity (17) also in other configurations.

For example, said integrated device can be inserted not perpendicularly to the direction of the flow, but inclined in the direction of the flow of the substantially plastic material.

Advantageously, the apparatus for measuring at least one “Px” property of a substantially plastic material can be used continuously and for a long period of time. Therefore a preferred method of the present invention is the continuous or semi-batch (semi-continuous) pyrolysis process of substantially plastic material.

To apply the method according to the present invention to the pyrolysis control of the substantially plastic material, it is necessary to have at least one calibration curve “Cx” capable of correlating the reflectance spectrum of the substantially plastic material fed to the pyrolysis reactor with the values of at least one parameter “Px” of said substantially plastic material.

Preferably said at least one property “Px” of said substantially plastic material is the hydrogen/carbon ratio (i.e. the H/C index, “H/C index”) and/or the carbon index (“C.I.” or “Carbon index”).

The carbon index and the hydrogen/carbon ratio (H/C index) are calculated according to the following formulae:

$$H / C Index = 100 \cdot \frac{12}{2} \cdot \frac{{\sum }_{materials} Weight atoms H}{{\sum }_{materials} Weight atoms C} \\ Carbon Index = 100 \cdot \frac{{\sum }_{materials} Weight atoms C}{{\sum }_{materials} Weight atoms ALL}$$

where the summation is carried out on each material of which the substantially plastic material is composed, and where “Weight atoms” means the total mass of the atom indicated in the material (or of all atoms for “All”, that is the weight of the material). If the quantity of each material in the substantially plastic material is not known, it is evident that it is in any case possible to calculate said ratios by knowing the weight of the hydrogen and carbon atoms of the substantially plastic material in its entirety. It is in fact simple to show that the two above calculation formulae are equivalent to the following two:

$$H / C Index = 100 \cdot \frac{12}{2} \cdot \frac{Weight atoms H}{Weight atoms C} \\ Carbon Index = 100 \cdot \frac{Weight atoms C}{Weight atoms ALL}$$

where said weights of the atoms are the weight of the atoms indicated in the substantially plastic material, i.e. considered in its entirety.

The calibration curve can be prepared with the methods known to the expert in the art. The calibration curve can be obtained by univariate regression methods. Preferably the calibration curve can be obtained by multivariate regression methods.

To obtain the calibration curve, for example, it is possible to prepare a plurality of substantially plastic materials (hereinafter referred to as “sample materials”), each of which is subjected to at least one primary analysis capable of determining the value of said at least a “Px” property. Therefore, according to an embodiment of the process for the pyrolysis of substantially plastic material of the present invention, said determination of the value of at least one property “Px” of said substantially plastic material is carried out by means of a correlation of said analytical measurement “Ax” with the analytical measurement obtained by acquiring a plurality of model absorbances of plastic materials having a known value of said at least one property “Px”.

For example, if the “Px” properties were the H/C index (“H/C ratio”) and the carbon index (C.I. or “carbon index”), it is sufficient to analytically determine the quantity total of the elements present in the substantially plastic material.

The H/C index would therefore be easily calculated from the ratio of the amount of hydrogen with respect to the amount of carbon, while the carbon index is easily calculated from the ratio of the amount of carbon with respect to the total weight.

For this purpose, for example, it is possible to use an elemental analyser which provides for a complete combustion of the sample followed by analysis of the gases produced by gas chromatography, thermal conductivity, infrared spectroscopy, or a combination of these techniques.

In general, the greater the number of sample spectra used to prepare the “Cx” calibration curve, the more accurate the determination of the associated “Px” parameter is.

According to the present invention, the number of sample materials used for the definition of the calibration curve is equal to at least 5, more preferably at least 10. In a particularly preferred embodiment, the number of sample materials is between 10 and 50.

Advantageously, said sample materials can be prepared by making mixtures of substantially plastic materials having the known property “Px”.

For example, said sample materials can be blends of polymers comprising polyethylene, polypropylene, polyvinyl chloride, polystyrene, polyurethane, polyethylene terephthalate and cellulose in various ratios. For example, the value of the H/C index and the carbon index value for said polymers is known, or in any case easily determined with the aforementioned techniques. Consequently it is possible to calculate said H/C index and carbon index also on the mixture, according to the given formula.

Alternatively, said sample materials can be different samples of the substantially plastic material obtained from the recycling of plastic material. Advantageously, it is advisable to take substantially plastic: materials of different origins and/or of different manufacturers.

According to the present invention, it is particularly preferred to make sample materials starting from substantially plastic materials obtained from the recycling of plastic material, and adding measured quantities of other polymers and/or non-polymeric substances, of which the property “Px” such as the value of the H/C index or the carbon index is known.

Advantageously, these polymers can be those already mentioned, i.e. polyethylene, polypropylene, polyvinyl chloride, polystyrene, polyurethane, polyethylene terephthalate and cellulose, while said non-polymeric substances can be for example wood, carbon black, graphite, calcium carbonate, talc.

The spectra on the sample materials which contain at least 50% by weight of meltable substances can be obtained by measuring the absorption of light in reflection after having brought the same materials at least partially in the molten state.

Furthermore, the temperature in the measurement conditions is preferably between 140 and 300° C., even more preferably between 16° and 260° C., and the pressure in the measurement conditions is preferably not less than 2 bara, even more preferably between 3 and 300 bara.

It has in fact been observed that these temperature and pressure conditions facilitate the obtaining of high repeatability spectra. For example, in low pressure conditions, for example sub-atmospheric, the presence of material in the gaseous state was observed. This can lead to spurious spectra, especially if the gas is developed in the measurement zone (for example, due to pressure reduction caused by pressure drop).

To obtain the spectra on which to carry out the calibration curve, it is preferable that these sample materials are processed so that the spectrum is read by the same apparatus which is then used in the process of the present invention and in similar process conditions.

Preferably, it is also advantageous to provide more than one sample spectrum for each sample material. Even more preferably, it is advantageous to make several sample spectra per sample material and under different process conditions.

In particular, it is preferred to make multiple sample spectra on the same sample material at different temperatures, pressures and flow rates of sample material.

In fact, the spectra can vary significantly according to said variables (temperature, flow velocity, pressure), and in particular according to the temperature. These variables that affect the spectrum obtained will be referred to hereinafter as “accessory variables”.

If not taken into account, the variations of these accessory variables can therefore affect the quality of the calibration curve made from these spectra.

However, the Applicant has discovered that it is possible to obtain an excellent calibration curve if said accessory variables are taken into account, either implicitly or explicitly, as described below.

There are no particular limitations on the spectrum meter that can be used. Preferably, said spectrum meter is a spectrophotometer. Even more preferably, said spectrophotometer is a Fourier transform (FTIR) diode series or dispersion spectrophotometer.

In all cases, the spectrum is typically obtained in digital form. In particular, typically the digital spectrum obtained includes the values of the spectrum for a discrete number of wave numbers, also called channels. Advantageously, in the case of the diode series spectrophotometer, said channels can correspond to the individual diodes.

Advantageously, the light radiation reflected by the sample analysed and collected by the detection system can be processed, according to the techniques known to the expert in the art, in the form of a reflectance spectrum (R) or preferably of absorption (A) as a function of the number d wave of the incident radiation (typically expressed in cm⁻¹) or as a function of the wavelength (typically expressed in nm). The absorption (A) is calculated starting from the measured value of the reflectance (R) based on the relationship A=log (1/R), where “log” is the natural logarithm.

Once acquired, the calibration and measurement spectra can be pre-processed with the methods known in the art to correct any spectral distortions due for example to displacements of the baseline.

To determine a given calibration curve “Cx”, the calibration spectra and the values of the property “Px” (already determined or otherwise known for the sample materials as explained above) are analysed with known statistical-mathematical methods of univariate linear regression and/or multivariate or in general by applying Machine Learning (ML) models such as neural networks (ANN, Artificial Neural Networks), genetic algorithms (GA, Genetic Algorithms), Fuzzy logic, particle swarm optimisation (PSO) and their combinations.

Preferably, the multivariate linear regression method is chosen from: multiple linear regression (MLR) method, partial least squares (PLS) method, principal component regression (PCR) method and their combinations.

According to one modality, the calibration curve resulting from the application of the aforementioned multivariate regression methods can be a linear combination of absorbances or other quantities derived from these.

Therefore, according to this modality, for each sample spectrum the following equation can be written, hereinafter referred to as “linear regression equation”:

$${Px}_{j} = {k}_{0} + {\sum }_{i = 1}^{N} {k}_{i} \cdot {A}_{ji} \\ j ∈ [ 1 , M ]$$

where:
“M” is the number of sample spectra evaluated;
“j” is the representative index of the sample spectrum “j” performed on a specific sample material “k” (as mentioned, preferably more sample spectra are evaluated for each sample material);
Pxⱼ is the value of the parameter Px for the sample material “k” used in the measurement of the sample spectrum “j”
“i” is the channel number
“N” is the number of channels, that is, the number of discrete wave numbers that make up the spectrum
Aⱼᵢ is the absorption of channel “i” (corresponding to the absorption at wavelength λi) measured on the spectrum “j”, or other quantity derived from the absorbance
kᵢ with i in the range from 0 to N are the coefficients of the Cx calibration curve to be found.

We therefore have M equations (one for each sample spectrum evaluated) in N+1 unknowns (the kᵢ coefficients).

If the number of sample spectra M is greater than N+1, and if there are no linearly dependent sample spectra (i.e. one linear combination of two or more other spectra), mathematically it is possible to regress the system of M equations obtaining the values of the kᵢ coefficients. This is the multiple linear regression (MLR) method.

However, it is preferable to reduce the number of unknowns N+1, because many unknowns are actually not linearly independent. For example, the signal relating to the absorption of the double bond of the carbon atom in ethylene has different upper harmonics (“overtone”), so the presence of this double bond increases the absorbance signal on different channels.

Therefore, according to one preferred modality of the present invention, the number of unknowns reduced by using multivariate analysis methods.

According to one first method, the data relating to the absorptions of the sample spectra are subjected to the analysis of the main components (PCA). Preferably, from 5 to 15 main components are extracted, still more preferably from 8 to 11 main components.

The model is then obtained with the linear regression equation defined above, where however “Aⱼᵢ” represents the value (“score”) of the main component “i” relative to the sample spectrum “j”. This way of applying PCA is called principal component regression (PCR).

According to a further, more preferred modality of the present invention, the data relating to the absorptions of the sample spectra and those of the parameter Pxⱼ are subjected to partial least squares regression (“partial least squares” or “PLS”).

The use of the information of the parameter Pxⱼ in the regression makes it possible to determine the main components capable of maximally describing the variability of the regressed parameter Pxⱼ.

As for the PCR, in this way preferably from 5 to 15 main components are extracted, even more preferably from 8 to 11 main components.

The model is then obtained with the linear regression equation defined above, where however “Aⱼᵢ” represents the value (“score”) of the main component “i” relative to the sample spectrum “j”, this time obtained by PLS.

As previously indicated, the Applicant has verified that the accessory variables have a significant effect on the result of the calculation of the regression coefficients.

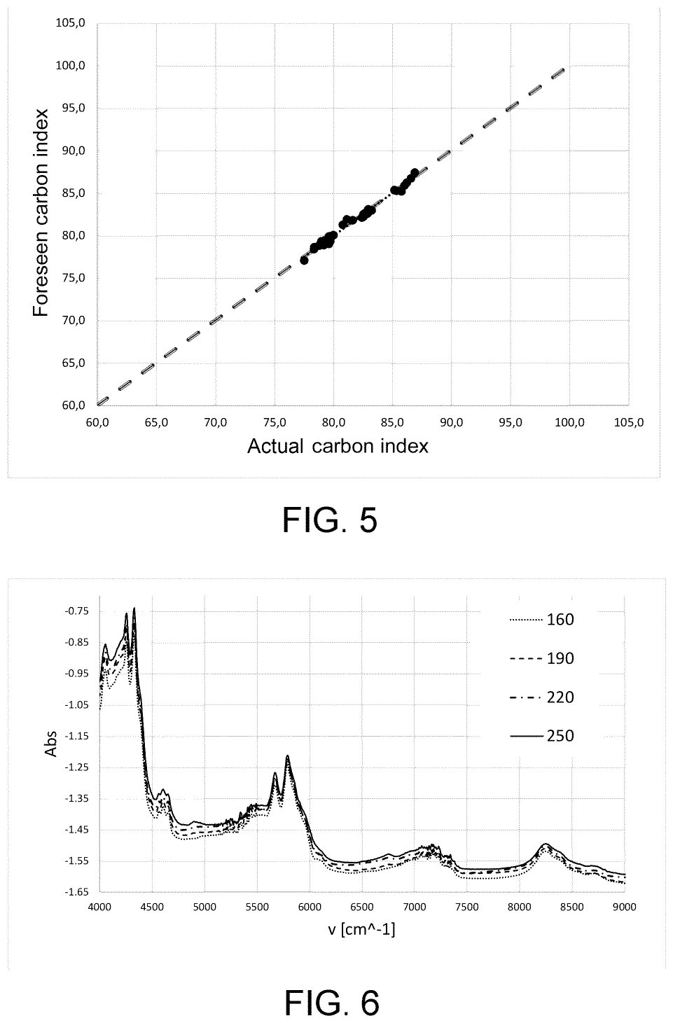

FIG. 6 reports different spectra obtained on the same sample material at different temperatures. There is evidently a significant temperature effect. For example, although the composition of the spectrum is not easily decipherable, it can be noted that the distance between the minima and maxima of absorbance tends to decrease as the temperature increases. Despite this relevant effect on the spectrum, the Applicant has discovered that, if multiple sample spectra are carried out for each sample material, by varying said variables so as to cover the interval that can be found in the pyrolysis process of the present invention at the time of the measurement of the absorption spectrum, then the multivariate regression methods such as PCR and/or PLS described above are able to implicitly remove the effect of temperature. In other words, the regression with respect to a small number of principal components as described above is able to select the combination of the absorbances of several channels capable of maximising the response with respect to the parameter Pxⱼ, which therefore limits the effect of temperature, which instead appears as residual noise. This is the implicit mode of reducing the effect of the temperature mentioned above.

Alternatively, according to an even more preferred modality, called explicit modality, it is possible to explicitly take into account the accessory variables.

According to this modality, the linear regression equation becomes:

$${Px}_{j} = {k}_{0} + {\sum }_{i = 1}^{N} {k}_{i} \cdot {A}_{ji} + {\sum }_{i = N + 1}^{N + NVA} {k}_{i} \cdot {VA}_{j} \\ j ∈ [ 1 , M ]$$

where the symbols correspond to what has already been described in the previous linear regression equation, apart from VAⱼ which is the accessory variable during the absorption measurement of the sample spectrum “j” and NVA which is the number of accessory variables used in the explicit model.

Preferably, said accessory variable is the speed (calculated as the ratio between the volumetric flow rate and the section of the hollow duct) and/or the temperature of the sample material, even more preferably it is the temperature of the sample material.

The model therefore requires additional unknown NVA. As with the implicit modality disclosed earlier, it is preferred to apply regression analysis on principal components (PCR), and more preferred is partial least squares regression (PLS).

Preferably, both according to the explicit and implicit modality, the number of process conditions applied on the sample material by varying at least one accessory same variable is at least 8, preferably between 12 and 100.

It is advantageous to produce more than one sample spectrum under the same process conditions (i.e. with accessory variables, for example temperature and flow rate, substantially constant).

Preferably, both according to the explicit and implicit modality, the number of sample spectra carried out under the same process conditions is at least 4, preferably from 8 to 20.

The “Cx” calibration curve obtained from multivariate regression analysis (for example, MLR, PCR or PLS) is subsequently validated using a series of spectra prepared in the same way as the sample spectra used to determine the “Cx”, i.e. made on substantially plastic material having the property “Px” known or determinable with a primary analysis as previously illustrated.

Surprisingly, a so-called “rotation” mode has been found which allows obtaining a calibration curve which is particularly effective in predicting the “Px” control parameters, in particular the carbon index and the H/C index.

According to this method:
A) the sample spectra are divided so that from 1 to 40% (in number) of the spectra, preferably from 10% to 30%, are used for validation, and the rest for calibration;
B) multivariate regression is performed, preferably PLS or PCR, on the spectra selected for calibration, carrying out the validation on the spectra selected for validation, calculating the mean quadratic error of the “Px” property;
C) the sample spectra are subdivided again so that from 1% to 40% (in number) of the spectra, preferably from 8% to 25%, are used for validation, and the remaining ones for calibration, selecting validation spectra between those that have not been previously selected for validation;
D) the multivariate regression is repeated on the new selection of spectra for calibration, and the validation on the new selection of spectra for validation;
E) a new loadings matrix is calculated, in which each element of the matrix corresponds to the average of the corresponding elements of the single load matrices obtained in steps B) and D).

Once validated, the calibration curve can be used to calculate the value of the control parameter “Px”, such as the H/C index or the carbon index, by applying it to a spectrum acquired in-line. If multivariate regression methods were used, from the discrete values of the absorbances it is possible to calculate the values (scores) of the same main components of the PCA and/or PLS previously identified during the determination of the “Cx” calibration curve (that is, using the same loadings matrix).

In the production phase (i.e. in the pyrolysis process according to the present invention) the spectrum analyser can be advantageously connected to a control system, such as for example a computer, a calculation server, a distributed control system (DCS), a programmable logic controller (PLC) or a “field programmable gate array” (FPGA).

The spectrum analyser is able to perform spectrum measurements in a very short time, typically less than one minute. The calculation of said at least one parameter Px by means of said at least one calibration curve Cx is also very fast, generally consisting of a relatively small number (for an electronic computer) of algebraic operations. The control system can also be extremely fast. Therefore, the entire sequence of operations (from the “Ax” analysis to the calculation of the “Ox” process parameter) can be carried out in a very short time, less than a minute, or even a few seconds.

According to the present invention, it is preferable to repeat the acquisition of spectra at a high frequency, preferably at least 10 every hour, preferably at least 30 every hour, even more preferably from 60 to 3600 every hour and even more preferably from 120 to 900 every hour.

According to the present invention, it is preferable to acquire a series of spectra in order to calculate an average parameter Px to be passed to the process controller for the calculation of the process parameter “Ox”. In this way, in fact, it has been observed that it is possible to better take into account the presence of any material not in the molten state in the hollow duct where the spectrum measurement is carried out.

Preferably, said substantially plastic material consists of compositions of different plastics. Even more preferably, said compositions of different plastics comprise at least polymers with a high H/C index such as for example polyethylene, polypropylene, polyamides, polymethyl methacrylate and polymers with a low H/C index such as polystyrene, polycarbonate, polyethylene terephthalate.

Alternatively, or in combination, said compositions of different plastics include high carbon index polymers such as polyethylene (including LDPE, LLDPE, HDPE), polypropylene, polystyrene, elastomers and low carbon index polymers such as polyamides, polymethyl methacrylate, polyethylene terephthalate, polyvinyl chloride and cellulose.

Preferably, said substantially plastic material is characterised by an H/C index (H/C index) equal to at least 70, preferably between 80 and 98, even more preferably between 85 and 96. Preferably, said substantially plastic material is characterized by an index of carbon (carbon index) equal to at least 55, preferably between 65 and 95, even more preferably between 75 and 90.

In certain embodiments, said substantially plastic material contains at least one non-plastic material in an amount comprised between 0.01% and 10% by weight with respect to the weight of the substantially plastic material, or in an amount comprised between 0.05% and 7.5%, or in an amount comprised between 0.2% and 5%. Said non-plastic material may include at least one of the following materials: paper, cardboard, wood, compost (as defined by IUPAC in “Terminology for biorelated polymers and applications (IUPAC Recommendations 2012)”, Pure Appl. Chem., Vol. 84, No. 2, pp. 377-410, 2012, DOI 10.1351/PAC-REC-10-12-04), metallic materials such as aluminium and iron, and/or inert materials.

Optionally, said substantially plastic material can contain inorganic fillers such as silica, titanium oxide, talc, coke, graphite, carbon black, calcium carbonate. In certain embodiments, said fillers can be present in quantities of 0.01-10%, preferably 0.1-5%, with respect to the total weight of the substantially plastic material. In certain embodiments, the substantially plastic material has a final inorganic residue (ash), measured according to the method described herein, is at least 0.01%, preferably between 0.1% and 20%, more preferably between 0.4 and 12%, even more preferably between 1.1% and 7%, with respect to the weight of the substantially plastic material.

Optionally, said substantially plastic material can contain brominated and chlorinated additives used to make the plastic material fireproof or in any case impart flame retardant properties. Examples of said additives are hexabromocyclododecane, decabromodiphenyloxide, polybrominated diphenyl ethers and brominated polymers such as brominated styrene-butadiene copolymers or brominated polystyrene.

Optionally, said substantially plastic material can contain non-halogenated additives used to make the plastic material fireproof or otherwise impart flame retardant properties, such as phosphorus and nitrogen compounds.

If the substantially plastic material contains one or more of said materials or substances, the pyrolysis process according to the invention is not negatively affected.

By “non-constant composition” we mean that the composition is variable between different production batches. Alternatively or in combination, the composition is not constant because even within the same batch there is variability of the composition, for example due to the stratification of the material. In fact, during transport there can be stratification, which generally determines an increase in the concentration on the bottom of the heavier and/or small-sized or powdery plastics, and on the top an increase of the lighter and/or large-sized plastics.

Alternatively, said substantially plastic material is not of constant composition because it is supplied by different manufacturers or suppliers. Each manufacturer can have different production specifications and/or different production processes, so the product obtained is different.

Preferably, said substantially plastic materials are also recycled.

Preferably, said substantially plastic materials also contain halogenated components in quantities ranging from 0.01% to 10% by weight with respect to the weight of the substantially plastic material.

Preferably said substantially plastic materials are obtained by a process of selection (sorting) of plastic material. Even more preferably, said substantially plastic materials are the residual substantially plastic material, that is the substantially plastic fraction which remains after having recovered some plastics, that is, after having selectively extracted some plastics from the substantially plastic material fed to the selection process. Selective extraction consists in the substantially homomaterial extraction (i.e. as monoplastic) of certain plastics. Typically, in a sorting process it is possible to extract substantially pure plastic streams (i.e. as monoplastic) of the polyethylene, polypropylene and polyethylene terephthalate components. In this preferred selection, the substantially residual plastic material is therefore the material that results after the extraction of said substantially pure plastics. This fraction is known in Italy by the term “Plas Compound” or “Plasmix”, which is defined as the “set of heterogeneous plastics included in post-consumer packaging and not recovered as individual polymers” (art. 1 law proposal Chamber Act no. 4502 of 18 May 2017).

This substantially plastic material can be further selected to eliminate non-recyclable materials or used as such. In particular, according to a preferred method, said substantially plastic materials, possibly obtained from a selection process (sorting) of plastic material as defined above, are pre-treated before being used in the pyrolysis process of the present invention.

This pre-treatment preferably comprises a washing adapted to remove at least part of the organic matter.

Preferably, said pre-treatment also comprises, alternatively or in combination, the elimination of non-organic solid particulate, such as ferrous material and crushed stone.

The fluid comprising hydrocarbons obtained from pyrolysis which is in the liquid state at 25° C. is also called pyrolysis oil.

### Preferred Methods of the Process of the Present Invention

According to a preferred method, said probe capable of emitting light consists of one or more optical fibres.

According to a further preferred method, said detection system optically coupled with said probe is also constituted by one or more optical fibres.

According to a preferred method, said probe capable of emitting light and said detection system optically coupled with said probe are integrated in a single device having both functions.

According to a further preferred method, in said integrated device, both said probe capable of emitting light and said detection system optically coupled with said probe are constituted by optical fibres.

In this mode, preferably the optical fibres relating to the probe capable of emitting light are placed at the centre of said integrated device while the optical fibres relating to the detection system optically coupled with said probe are placed in the periphery, preferably equally spaced along a circumference passing through said centre.

According to an embodiment, said apparatus for measuring at least one property “Px” of a substantially plastic material also comprises a means for measuring the temperature of the substantially plastic material.

Said means for measuring the temperature can be any means known in the art, such as a thermocouple, a thermoresistance (such as a “PT100” or a “PT1000”), or an infrared meter.

Advantageously, the minimum distance between the temperature reading area and the area where the reflection spectrum is measured is not more than 100 mm, preferably not more than 30 mm, even more preferably not more than 3 mm.

Preferably, the means for measuring the temperature is a thermocouple and said integrated device comprising said probe capable of emitting light and said detection system optically coupled with said probe further comprises said thermocouple.

It has in fact been observed that the absorption spectra detected by said detection system optically coupled with said probe for detecting the reflected light depend on the temperature of the substantially plastic material illuminated by said light, and that if the temperature measurement is integrated in said device there is a better repeatability of the measurement.

Preferably, the substantially plastic material fed to the reactor in step a) is brought at least partially to the molten state by heating in a preheating apparatus. Said preheating apparatus can be a single screw extruder, a twin screw extruder or an auger. Said preheating equipment can be equipped with degassing for the evacuation of water vapour and any other gases produced, such as in particular hydrogen chloride (HCl).

For this purpose, it may be advantageous to feed said preheating equipment, in addition to said substantially plastic material, also additives capable of favouring the evolution of hydrochloric acid or to salify it. Such additives are preferably composed of the elements of group IA and IIA. Even more preferably are the oxides, hydroxides, carbonates, silicates and aluminosilicates of groups IA and IIA. Even more preferably they are calcium oxide, calcium hydroxide, calcium carbonate, sodium oxide, sodium hydroxide, sodium carbonate, potassium oxide, potassium hydroxide, potassium carbonate or sodium aluminosilicate.

The preheating temperature can be between 120 and 430° C., preferably between 150 and 320° C., even more preferably between 18° and 220° C. The residence time in said preheating apparatus is preferably less than 10 minutes, even more preferably less than 2 minutes, in particular less than one minute.

Therefore, according to a preferred method of the process of the present invention, said substantially plastic material at least partially in the molten state is obtained by means of a preheating apparatus, preferably an auger or an extruder. Said process for pyrolysis of substantially plastic material to obtain at least hydrocarbons that are in the liquid phase at 25° C. can be carried out both in batch mode, in continuous mode, and in semi-continuous mode.

In the latter mode, the substantially plastic material is loaded continuously, the generated vapours are continuously extracted, but any solid residue is kept inside the pyrolysis reactor. When the quantity of solid residue inside the reactor rises above a certain threshold, or at predefined time intervals, for example with a frequency ranging from 2 to 10 days, the material contained in the reactor is removed and then of said solid residue.

Preferably, the reactor is managed in continuous or semi-continuous mode, even more preferably in semi-continuous mode.

The pyrolysis process of the present invention is not limited by a particular type of reactor.

In particular, horizontal or vertical, stirred or non-stirred reactors, rotary reactors (kiln reactor), or screw reactors can be used.

Among the continuously stirred reactors (CSTR) it is possible to use totally filled reactors and reactors in which a separation of the gaseous phase from the phase that includes the liquid and other possible phases such as the solid (char) produced, or the reactors in which there is it is a free surface.

Preferably the reactor is a stirred reactor with a free surface.

According to one preferred method, the pyrolysis vapours produced by the pyrolysis reactor are subsequently passed through at least one condensation separator, so as to recover at least hydrocarbons that are in the liquid phase at 25° C. (as defined in the present invention).

By condensation separator (separator by condensation) we mean any equipment which receives a fluid in the gaseous state and is capable of removing sufficient heat from said fluid so as to generate at least a part of fluid in the liquid state. Examples of equipment are condensers comprising coils inside which a heat carrier fluid flows, capable of removing the heat from the processed gaseous fluid.

Other methods of heat removal can also be used, for example, alternatively or in combination, the condensation separator can be equipped with a jacket in which said heat transfer fluid capable of removing heat flows.

Advantageously, the flooding condenser can also be used, in which the condenser is partially flooded by the liquid phase produced, and whose condensing power is regulated by varying the height of said liquid phase, since only the coil which is not flooded is able to absorb calories from the steam to be condensed. This therefore allows effective adjustment of the capacitor power.

Alternatively, the condensation separator can consist of a distillation column. In this case, the condensed fluid originates in the column condenser and the condensed liquid flows back by gravity or by pumping into the column, condensing the vapours which are inside the column.

By using a condensation separator of the distillation column type, a better fractionation of the incoming vapours is also obtained, i.e. the separation between higher boiling components which are condensed and lower boiling components which remain in the vapour phase, as each equilibrium stage allows enrichment of the liquid phase of heavy components and an enrichment of the gas phase of light components. In addition, the condensed liquid which falls inside the column carries out a washing of the vapours inside the distillation column. This has the result of retaining any solid particulate present in the incoming vapours, which ends up being collected in the liquid phase.

Any technique known in the art can be used to maintain the pressure in the pyrolysis reactor at a defined value.

According to a first method, the pressure can be maintained at a defined value by regulating the heat extracted from the condensation separator located downstream of the reactor and in fluid connection with it.

According to a second method, the pressure can be regulated with a controllable pressure drop device located downstream of the pyrolysis reactor and/or downstream of said at least one condensation separator.

Said controllable pressure drop device can preferably be of the concentrated pressure drop type such as for example a lamination valve, or with distributed pressure drop (tube with variable section) or a barometric device (such as for example a hydraulic seal).

Non-condensed gases, including the auxiliary gas possibly used for pressurization, can be sent to a thermo-oxidation system before being released into the atmosphere.

Advantageously, the two control systems (regulation of the power of the separator by condensation and regulation on the controllable pressure drop device) can be combined together.

The pressure in the reactor is preferably kept within a range between atmospheric pressure and 13 bara. More preferably, said pressure is kept within a range of between 1.1 and 8 bara. Even more preferably, said pressure is kept within a range of between 1.5 and 6 bara. Most preferably, said pressure is kept within a range between 1.1 and 4 bara or between 2.5 and 4 bar.

The pressure in the reactor can be measured according to any method known in the art. For example, pressure transducers can be used, placed inside the reactor. Alternatively, according to a preferred method in the case in which inert gases such as nitrogen are used for the initial pressurization of the reactor, the pressure sensor can be advantageously placed inside the injection duct of said inert gases, even more preferably towards the entered the reactor.

Advantageously, the determination of the value of the property “Px” of said substantially plastic material by means of said at least one analytical measurement “Ax” is used to set at least one parameter “Ox” of the pyrolysis process.

Said at least one parameter “Ox” is preferably at least one of the following parameters: the pressure of the pyrolysis reactor, the temperature of the pyrolysis reactor, the residence time of the substantially plastic material in the pyrolysis reactor and the flow rate of the substantially plastic material in the pyrolysis reactor and a combination of said parameters.

The setting (“set point”) of said at least one “Ox” parameter is calculated based on the determination of the value of said at least one “Px” property.

Said calculation can advantageously be a simple expression. If at least one property “Px” is more than one, said expression for the calculation of at least one parameter “Ox” can advantageously include more than one property “Px”.

The adjustment of the “Ox” process parameter can be carried out with any means known in the art, for example by means of a controller capable of reading said “Ox” value, comparing it with the set value (“set point”), and acting on at least one parameter of at least one plant element (such as those already disclosed previously) in order to bring the difference between said two values to zero. For this purpose, any process controller can be used, such as a PID controller, with fuzzy logic, with particle swarm optimization (PSO) or with neural networks, or combinations of these, such as a controller Integrated PID with fuzzy logic controller.

Preferably, said regulation is carried out with algorithm (proportional, integrative, derivative), in positional (position PID) or velocity (velocity PID) form.

Preferably, the liquid product at 25° C. condensed by said pyrolysis vapours (i.e. the pyrolysis oil) obtained by the present invention has a C5-C12 fraction equal to at least 35%, and at the same time a C21-and-higher fraction (hereinafter denominated with: “C21+”) equal to at most 3.5%. Preferably, the pyrolysis oil obtained from the process of the present invention is a mixture that includes hydrocarbons in an amount greater than 90% by weight with respect to the total weight of the mixture.

Preferably, the yield in C5-C12 obtained by the present invention is at least 30% while the yield in C21-and-higher fraction is equal to at most 3%.

The applicant of the present invention has observed that it is advantageous to define an Overall Index (hereinafter also referred to as “Overall Index” abbreviated “O.I.”). This index is defined as the Carbon Index (C.I.) multiplied by the H/C index (as defined in the present invention) divided by 10000:

$$O . I . = \frac{[ C . I . ] \cdot [ H / C idx ]}{10000}$$

According to a preferred method of the present invention, the “Ox” parameter is determined by an expression which is a function of the Overall Index (O.I.).

According to a particularly preferred method of the present invention, the “Ox” parameter is the pyrolysis pressure and said pyrolysis pressure, aka “Ox”, is set at a pressure equal to at least a threshold pressure PS when said “Overall index” O.I. results greater than or equal to 0.7, and at a pressure lower than said threshold pressure when said “Overall index” O.I. is less than 0.7. Preferably said threshold pressure PS is at least 1.5 bara, even more preferably between 2 and 2.9 bara, in particular 2.5 bara. Generally this mode allows to achieve the best results.

In fact, by applying this criterion in Examples 1 to 11 of the invention, a yield in C5-C12 of at least 30% was obtained, and at the same time a yield of C21-and-higher fraction (C21+) equal to at most 3%.

### Example of Apparatus for the Procedure of the Invention

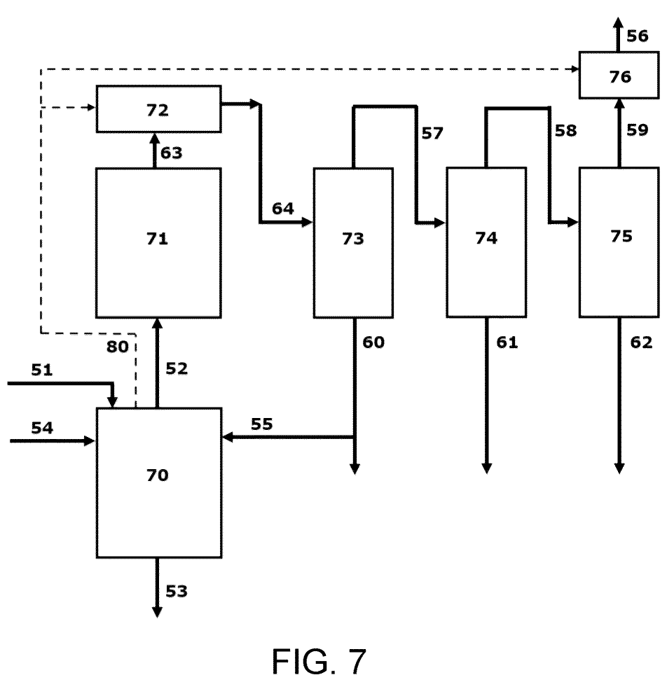

FIG. 7 schematically illustrates an example of an apparatus for the process of the invention, in which the following are visible:
a reactor (70) for the pyrolysis of substantially plastic material (54) which produces pyrolysis vapours (52) and a solid residue (53), and which optionally receives an auxiliary gaseous fluid (51) to facilitate the maintenance of the pressure inside the reactor;
a second reactor (71) which subjects the vapours to pyrolysis (52) coming from the pyrolysis reactor (70) to a thermal or thermocatalytic treatment;
a first pressure control device (72), for example a valve, which acts in feedback with respect to the pressure value (80) measured in the pyrolysis reactor (70);
a first condenser (73) whose condensates (60) are partially returned (55) to the pyrolysis reactor (70);
a second condenser (74) which receives the vapours (57) coming from the first condenser (73) producing a second condensate (61) and the vapours (58);
a third condenser (75) which receives the vapours (58) from the second condenser (74) producing a third condensate (62) and non-condensed vapours or the residual gas (59);
a second device for controlling the pressure (76) in feedback with respect to the pressure value (80) measured in the pyrolysis reactor (70), for example a valve that restricts the passage section of the residual gas leaving the condenser (59) before sending the residual gas (56) to the unit capable of receiving it.

### Examples of Implementation of the Pressure Control According TO THE INVENTION

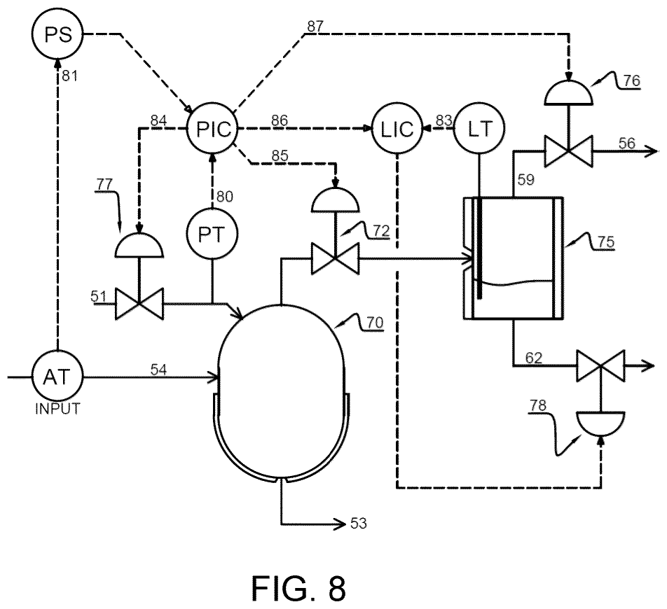

FIG. 8 illustrates some examples of embodiment of the pressure control according to the invention, showing a condensation separator of the flooding type (75) equipped with a level sensor (LT) and a level adjustment system by modulating the opening valve (78) on the condensates outlet (62).

The pyrolysis reactor (70) receives at its inlet the substantially plastic material (54) and optionally an auxiliary gaseous fluid (51), producing a solid residue (53) and pyrolysis vapours directed towards the at least one condensation separator (75). The optional regulation valve (72) receives the pyrolysis vapours from said pyrolysis reactor (70) and sends them to a condensation separator (75). The opening adjustment is given by the signal (85).

The condensation separator (75), in FIG. 8, is a flooded condenser: the condensed fluid floods the lower part of the condenser, and condensation is carried out by passing a heat transfer fluid, colder than the pyrolysis vapours, into a jacket or coil positioned so that depending on the level of the condensed liquid the part of the jacket in contact with the vapours to be condensed varies (for example, by applying the jacket on the side wall of said condenser).

The optional regulation valve (76) adjusts the pressure by restricting the passage section of the residual gas (59) before sending it (56) to the receiving unit.

The regulation valve (78), optional, regulates the outflow of the condensed fluid (62) and therefore the flooding level of the flood condenser (75).

The optional regulation valve (77) regulates the flow rate of the auxiliary gaseous fluid entering the pyrolysis reactor (70).

The level controller (LIC) reads the level signal (83) of the flooded condenser (75) measured by the level sensor (LT), and adjusts in feedback the opening of the valve (78) to ensure that the level (83) corresponds to the set point indication (86) received from the PIC controller. It should be noted that said set point indication (86) is equal to 0 for 100% level (i.e. maximum flooding=minimum condensing power) and 100 for 0% level (i.e. empty condenser=maximum condensing power).

The opening indication (87) sent to the valve (76) is 0 for closed valve and 100 for fully open valve.

The opening indication (84) sent to the valve (77) instead operates in reverse mode, because the valve (77) must open to increase the pressure of the reactor (80) and close to decrease it.

The pressure signal of the pyrolysis reactor (80) can be the result of the processing of multiple pressure transducers, as explained below; moreover, as shown in the figure, it can be detected on a clean fluid sent to the pyrolysis reactor, near the outlet towards said reactor, so that the membrane of the transducer remains clean. The Figure shows the case in which said pressure signal is taken on the conduit which carries the auxiliary gaseous fluid (51) to the pyrolysis reactor.

The pressure set point of the pyrolysis reactor (PS) can be locally, or provided manually, for example by setting the value on the control panel of the plant, or it can be remotely, or come from an external setting signal.

Said external signal can be a set point (81) calculated on the basis of one or more parameters read on the substantially plastic material arriving in the pyrolysis reactor (54). For example, said pressure set point can be an expression in which the variables are the H/C index (H/C index) and the carbon index (carbon index) of said substantially plastic material, measured by an analyser in line or offline (AT INPUT).

The pressure controller (PIC) reads said pressure signal (80) and compares it with the set point (PS), and acts individually on one of the regulation devices (84, 85, 86, 87) or in combination, for example using a PID algorithm (proportional, integrative, derivative) in feedback, in order to minimise the error between the signal read (80) and the set point (PS). One example of embodiment of said combination is obtained by using the adjustment devices (86) and (87) in split range mode.

### Method of Gas-Chromatographic Analysis on Pyrolysis Oil Samples

The pyrolysis oil samples were characterised by gas chromatographic analysis. The qualitative identification of the compounds was preliminarily carried out using the coupled gas chromatography-mass spectrometry (GC-MS) technique, while the quantification of the same was carried out by gas chromatography with flame ionization detector (GC-FID). Below are the instrumental parameters adopted for the GC-FID analysis:
GC: Agilent HP 7890 B, equipped with MPS Gerstel autosampler
Column: HP-PONA Agilent Technologies J&W-50 m-0.2 mm-0.5 μm,
Carrier (H2): 1.1 mL/min constant flow
Injector: 320° C., 255:1 split, 3 mm liner (Ultra Inert) with glass wool
Detector: 360° C.
Oven: Column temperature program: 20° C. 5 min, in 2° C./min up to 70° C. for 5 min, in 2° C./min at 160° C. for 5 min, in 2° C./min at 320° C. for 30 min (Run time: 195 min).

The samples are analysed as such by attributing an arbitrary response factor equal to one for all the compounds; the concentrations obtained are then normalised to 100%.

### Method of Gas-Chromatographic Analysis on Wax Samples

By wax we mean the fraction left on the bottom after the ultracentrifugation of the pyrolysis oil, as described below.

This fraction is analysed in different ways to allow the identification of compounds with a high molecular weight.

In fact, these compounds could plausibly not be eluted and analysed during gas chromatographic analyses.

Before taking the sample for GPC analysis, the pyrolysis oils contained in Schott bottles were heated to 50° C. to homogenize their contents (in some cases characterized by deposits and/or stratification of waxy compounds at room temperature or chilled). A few mg of sample in 1,2,4-trichlorobenzene (Baker) added with 10 μL of n-heptane (internal marker) were dissolved under heat (one hour of dissolution at 150° C.) in order to obtain a concentration of approximately 1.8 mg/mL.

The analyses were carried out on a chromatographic apparatus consisting of:
High temperature GPC-IR Polymer Char
bench of 3 TSK gel HT2 columns with 13 μm dimension and pre-column
infrared detector IR5 at high temperature that provides an absorbance signal proportional to the quantity of methyl and methylene groups.

The experimental conditions adopted are as follows:
eluent: 1,2,4 TCB stabilized with BHT
flow: 1 mL/min
temperature: pump at 25° C., injector at 150° C., columns at 150° C., detector at 150° C.
injection volume: 200 microliters
internal standard: n-heptane.

### Method of Gas-Chromatographic Analysis on Pyrolysis Gas

The pyrolysis gas effluent samples were sampled in 500 mL Swagelok cylinders of the DOT type (i.e. regulated by the U.S. Department of Transportation-DOT) in 304L stainless steel, internally coated with PTFE to make the internal surface inert. The instrumentation used is an Agilent 490 μGC equipped with 3 modules in parallel, each of which determines only certain types of compounds. In particular:
Module 1: 10 m MS 5 Å with heated injector and backflush
Module 2: 10 m PPQ with unheated injector
Module 3: 10 m CpSil-5CB with heated injector.

Below are the instrumental parameters adopted for the various modules:
Module 1: T injector: 110° C., Backflush: 30 s, t injection 100 ms, T column: 45° C., Carrier gas pressure: 80 kPa, Carrier gas: Argon (essential for hydrogen analysis).
Module 2: injection t: 15 ms, column T: 70° C., carrier gas pressure: 180 kPa, carrier gas: helium.
Module 3: T injector: 110° C., t injection: 20 ms, T column: 70° C., Carrier gas pressure: 230 kPa, Carrier gas: helium.

Each module analyses only a few specific compounds:
Module 1: Hydrogen, oxygen, nitrogen, methane, CO.
Module 2: CO2, ethylene, ethane, propylene, propane, propadiene, propyne, i-butane, i-butene, 1-butene, 1,3-butadiene, n-butane, trans-2-butene, cis-2-butene.
Module 3: 1-buten-3-ino, 1,2-butadiene, i-pentane, 1,4-pentadiene, 1-pentene, n-pentane, 2-methyl-2-butene, 1,3-pentadiene, cyclopentene, n-hexane, methyl-1,3-cyclopentadiene, benzene, 3-ethylcyclopentene, methylcyclohexane, toluene, ethylbenzene, xylene.

The quantification is carried out by means of a calibration line with an external standard, consisting of two calibration cylinders with the following composition:
Tank 1: PENTENE-2 (trans)=0.1% mol; PENTENE-2 (cis)=0.1% mol; PENTENE-1=0.1% mol; PENTANE-n=0.25% mol; METHYL-2 BUTENE-2=0.2% mol; ISOPENTANE=0.5% mol; HEXANE-n=0.1% mol; PROPYLENE=20% mol; PROPANE=0.5% mol; PROPADIENE=0.5% mol; METHANE=20% mol; ISOBUTENE=1% mol; ISOBUTANE=0.5% mol; HYDROGEN=15% mol; ETHYLENE=30% mol; ETHANE=3% mol; CARBON OXIDE=1% mol; CARBON DIOXIDE=0.5% mol; BUTENE-1=1% mol; BUTENE-2 (trans)=0.5% mol; BUTENE-2 (cis)=0.5% mol; BUTANE-n=0.5% mol; BUTADIENE-1.3=1.5% mol; ACETYLENE=0.5% mol; Complement to 100%: NITROGEN. Volume of the cylinder [liters]: 40; Charging pressure [bar]: 6.29; Type of cylinder: Aluminium.
Tank 2: BENZENE=0.0302% mol; TOLUENE=0.0323% mol; METHYCLICLOHEXANE=0.0674% mol; STYRENE=0.0334% mol; ETHYLBENZENE=0.0339% mol; Complement to 100%: HELIUM. Volume of the cylinder [liters]: 5; Charging pressure [bar]: 13.9; Type of cylinder: Aluminium.

The following compounds are not present in the calibration cylinders. The calibration of compounds sufficiently similar to them was therefore used, which have very similar response factors (the difference in this case is negligible):

| Compound: | Calibrated with: |
|---|---|
| propyne | Propadiene |
| 1-buten-3-ino | 1,3-butadiene |
| 1,2-butadiene | 1,3-butadiene |
| 1,3-pentadiene | 1,3-butadiene |
| 1,4-pentadiene | 1,3-butadiene |
| Cyclopentene | 1-pentene |
| Methyl-1,3- cyclopentadiene | 1-pentene |
| 3- methylcyclopentene | 1-pentene |

### Method of Thermogravimetric Analysis (TGA) on Solid Residue (CHAR)

The TGA analysis was performed on an instrument of the TA Instrument model Q 500. The temperature calibration was carried out using the Curie Point of Alumel and Nickel samples, while the weight calibration was carried out using certified weights supplied by TA Instrument together with the analyser. The sample as it is, weighed for quantities between 20-30 mg in a stainless steel sample holder, was placed together with the sample holder on the platinum crucible of the TGA analyser. The use of the stainless steel sample holder facilitates the isolation and recovery of the final residue (ashes), preserving the integrity of the platinum crucible. The sample is subjected to an analytical procedure in three steps:
1^{st} step (pyrolysis in nitrogen atmosphere): starting from an initial temperature of 40° C., the sample is heated at a controlled rate (v=10°/min) up to 800° C.
2^{nd} step (cooling in nitrogen atmosphere): starting from an initial temperature of 800° C., the sample is cooled at a controlled rate (v=20° C./min) up to 400° C.
3^{rd} step (thermo-oxidation in air atmosphere): starting from a temperature of 400° C., the sample resulting from pyrolysis (step 1) is subjected to heating at a controlled rate (v=20° C./min) until at 850° C.

The integrations were performed using Universal software (TA Instruments), resulting in:
STEP 1: the weight loss at various temperatures after determining the temperature corresponding to the maximum peak of the derivative of the weight loss with respect to the temperature and the residue at 800° C.
STEP 3: the weight loss at various temperatures after determining the temperature corresponding to the maximum peak of the derivative of the weight loss with respect to the temperature and the residue at 850° C. For STEP 3 the weight losses correspond to one or more carbonaceous species different in allotropic state or in particle size.

Some illustrated but non-limiting examples of the present invention follow.

### Examples

### Raw Material

It was considered appropriate to use both some samples of substantially recycled plastic material and virgin raw material (i.e. not coming from recycling but from synthesis). The substantially recycled plastic material used is Plasmix, which is the set of heterogeneous plastics included in post-consumer packaging and not recovered as individual polymers in said pre-treatment plants of a plastic recycling plant. In particular, four Plasmix samples with different H/C index and different carbon index were selected.

Virgin raw material was also used, the composition of which is therefore known and constant to facilitate the repeatability of the experiments. Furthermore, by combining recycled raw material with virgin raw material it was possible to expand the base of polymer blends evaluated, in order to increase the range of H/C ratios and carbon index used.

The virgin materials used were the following:

| Polymer | Initials | Grade | Manufacturer |
|---|---|---|---|
| Polyethylene | LDPE | Riblene ® | Versalis |
| low density | | FC20 | |
| Linear low density | LLDPE | Flexirene ® | Versalis |
| polyethylene | | CL10 | |
| Polyethylene high | HDPE | Eraclene ® | Versalis |
| density | | BC82 | |
| Polypropylene | PP | Isplen ® | Repsol |
| | | PP040 | |
| Polystyrene | PS | Edistir ® | Versalis |
| | | N3782 | |
| Polyethylene | PET | Monflakes ® | Montello |
| terephthalate | | R-PET | |
| Cellulose | CELL | C6288 | Sigma-Aldrich |
| Polyvinyl chloride | PVC | S3160 | Vinnolit |

The polyethylene granules were mixed in the following ratio: 5.7% of HDPE Eraclene BC82, 34.3% of LLDPE Flexirene CL10 and 60% of Riblene FC20. This mixture is therefore the “PE” material used subsequently.

The following table shows the atomic composition (percentages by weight) of the materials used.

| | | MATERIALS (in initials) | | | | | |
|---|---|---|---|---|---|---|---|
| | | PE | PE | PS | PET | CELL | PVC |
| ATOM | H | 14.3% | 14.3% | 7.7% | 4.2% | 6.2% | 4.8% |
| | C | 85.7% | 85.7% | 92.3% | 62.5% | 44.4% | 38.7% |
| | N | 0.0% | 0.0% | 0.0% | 0.0% | 0.0% | 0.0% |
| | O | 0.0% | 0.0% | 0.0% | 33.3% | 49.4% | 0.0% |
| | Cl | 0.0% | 0.0% | 0.0% | 0.0% | 0.0% | 56.5% |

The following compounds were prepared using the listed virgin raw materials (parts by weight):

| | COMPOUND | | | | |
|---|---|---|---|---|---|
| | PAT1 | PAT2 | PAT3 | PAT4 | PAT5 |
| PE | 70.00 | 51.60 | 46.60 | 42.10 | 52.58 |
| PP | 30.00 | 22.20 | 20.00 | 18.00 | 22.55 |
| PS | 0.00 | 2.50 | 27.00 | 5.30 | 8.70 |
| PET | 0.00 | 2.50 | 5.40 | 26.50 | 8.60 |
| CELL | 0.00 | 20.20 | 0.00 | 7.10 | 6.83 |
| PVC | 0.00 | 1.00 | 1.00 | 1.00 | 0.75 |

On the basis of the content of carbon and hydrogen atoms of the raw materials used (indicated in the previous table), the carbon index and the hydrogen/carbon ratio (H/C index) were calculated, according to the equations provided in precedence.

For the 5 PAT1, PAT2, PAT3, PAT4 and PAT5 compounds used, the following H/C index and carbon index values can therefore be calculated:

| | COMPOUND | | | | |
|---|---|---|---|---|---|
| | PAT1 | PAT2 | PAT3 | PAT4 | PAT5 |
| H/C index | 100 | 95 | 83 | 83 | 90 |
| C.I. | 86 | 76 | 86 | 77 | 81 |
| Carbon Index | | | | | |
| H/C HIGH (>90) | + | + | − | − | 0 |
| Carbon Index | + | − | + | − | 0 |
| HIGH (>80) | | | | | |

Said H/C ratios and carbon index were also calculated on the substantially plastic Plasmix recycled materials B01, B02, B03 and B04 used in the experimentation:

RECYCLING MATERIAL

| | B01 | B02 | B03 | B04 |
|---|---|---|---|---|
| H/C index | 95.10 | 90.56 | 87.73 | 70.27 |
| C.I. | 82.90 | 79.60 | 82.50 | 78.80 |
| Carbon Index | | | | |
| H/C HIGH (>90) | + | + | − | − |
| Carbon Index | + | − | + | − |
| HIGH (>80) | | | | |

Analysing the two tables, it can be seen that PAT1 and PAT2 are compounds characterized by a high H/C index, PAT3 and PAT4 by a low H/C index; while PAT1 and PAT3 have a high carbon index (both 86) and PAT2 and PAT4 a low carbon index (both 76).

In this way it is possible to analyse the behaviour of the pyrolysis materials with respect to the two indicated variables (H/C index and carbon index).

Also in the recycled materials, two (B01, B02) are characterized by a high H/C index while the other two (B01, B03) are characterized by a high carbon index.

The PAT 5 compound is the average composition of the previous 4 (PAT1, PAT2, PAT3 and PAT4) and it was used to validate the model and experimentation.

Compounds Used to Produce the Calibration Curves and to Validate them

The following table illustrates the composition of the compounds prepared and used to prepare the calibration curves and to check them.

In the table, the “Compound” column indicates the name of the compound; “Base” indicates the base used. The percentages are calculated with respect to the base weight used only and not to the whole weight of the compound. For example, the “M03” compound is made with B01+4% LDPE, that is, with a compound that includes 100 parts of B01 and 4 parts of LDPE.

| Com- | | | | | | | | H/C | |
|---|---|---|---|---|---|---|---|---|---|
| pound | Base | LDPE | PS | PP | PVC | CELL | PET | idx | C.I. |
| M01 | B01 | | | | | | | 82.9 | 82.9 |
| M02 | B01 | 2% | | | | | | 82.9 | 82.9 |
| M03 | B01 | 4% | | | | | | 82.9 | 83.0 |
| M04 | B01 | | | | | | | 82.9 | 82.9 |
| M05 | B01 | | 1% | | | | | 82.9 | 83.0 |
| M06 | B01 | | 4% | | | | | 82.9 | 83.2 |
| M07 | B01 | | | | | | | 82.9 | 82.9 |
| M08 | B01 | | | 1% | | | | 82.9 | 82.9 |
| M09 | B01 | | | 3% | | | | 82.9 | 83.0 |
| M10 | B01 | | | | | | | 82.9 | 82.9 |
| M11 | B01 | | | | 1% | | | 82.9 | 82.4 |
| M12 | B01 | | | | 4% | | | 82.9 | 81.1 |
| M13 | B02 | | | | | | | 82.9 | 79.6 |
| M14 | B02 | 1% | | | | | | 82.9 | 79.7 |
| M15 | B02 | 3% | | | | | | 82.9 | 79.8 |
| M16 | B02 | | | | | | | 82.9 | 79.6 |
| M17 | B02 | | 1% | | | | | 82.9 | 79.7 |
| M18 | B02 | | 3% | | | | | 82.9 | 80.0 |
| M19 | B02 | | | | | | | 82.9 | 79.6 |
| M20 | B02 | | | | | 4% | | 82.9 | 79.0 |
| M21 | B02 | | | | | | | 82.9 | 79.6 |
| M22 | B02 | | | | | | 2% | 82.9 | 79.2 |
| M23 | B02 | | | | | | 4% | 82.9 | 78.9 |
| M24 | B02 | | | | | | | 82.9 | 79.6 |
| M25 | B02 | | | 2% | | | | 82.9 | 79.7 |
| M26 | B02 | | | 4% | | | | 82.9 | 79.8 |
| M27 | B02 | | | | | | | 82.9 | 79.6 |
| M28 | B02 | | | | 1% | | | 82.9 | 79.2 |
| M29 | B02 | | | | 3% | | | 82.9 | 78.4 |
| M30 | B03 | | | | | | | 82.9 | 82.5 |
| M31 | B03 | 1% | | | | | | 82.9 | 82.6 |
| M32 | B03 | 4% | | | | | | 82.9 | 82.7 |
| M33 | B03 | | | | | | | 82.9 | 82.5 |
| M34 | B03 | | 2% | | | | | 82.9 | 82.7 |
| M35 | B03 | | 4% | | | | | 82.9 | 82.9 |
| M36 | B03 | | | | | | | 82.9 | 82.5 |
| M37 | B03 | | | 1% | | | | 82.9 | 82.6 |
| M38 | B03 | | | 4% | | | | 82.9 | 82.7 |
| M39 | B03 | | | | | | | 82.9 | 82.5 |
| M40 | B03 | | | | 2% | | | 82.9 | 81.6 |
| M41 | B03 | | | | 4% | | | 82.9 | 80.8 |
| M42 | B04 | | | | | | | 82.9 | 78.8 |
| M43 | B04 | | | | 1% | | | 82.9 | 78.3 |
| M44 | B04 | | | | 3% | | | 82.9 | 77.5 |
| M45 | | 89% | 11% | | 0.50% | | | 82.9 | 86.2 |
| M46 | | 83% | 15% | | 2% | | | 82.9 | 85.8 |
| M47 | | 79% | 20% | | 1% | | | 82.9 | 86.6 |
| M48 | | 94% | 5% | | 1.50% | | | 82.9 | 85.3 |
| M49 | | 74% | 25% | | 1% | | | 82.9 | 86.9 |
| M50 | | 92% | 8% | | | | | 82.9 | 86.0 |
| M51 | | 96% | 2.50% | | 1.50% | | | 82.9 | 85.2 |

### Apparatus Used to Determine the Calibration Curves, and Subsequently Used to Feed the Pyrolysis Reactor, and Process Conditions

The apparatus in question consists of a ZSK 26 co-rotating twin-screw extruder with screw length ratio L to diameter D equal to L/D=32. The extruder is equipped with:
feeding section, equipped with a hopper and a screw profile with transport elements;
melting and mixing section, in which kneading and mixing elements are used, plus a left profile element to ensure filling;
degassing section, where the pressure of the polymer melt is reduced by reducing the diameter of the screw core and an opening is made in the cylinder, connected to a vacuum pump, for the suction of any gases produced;
Pressurisation section, where the diameter of the core of the screw is increased.

The extruder is equipped with a heating and cooling control system to regulate the temperature of the cylinder in the sections of the extruder indicated above.

The extruder outlet is connected to a hollow duct of the type indicated in FIG. 1, where an integrated device is connected including both a probe for the emission of light and said optically coupled detection system. The integrated device used for this purpose is a titanium reflection probe with sapphire window and optical fibres (7 of 400 micrometres plus another 7 of 600 micrometres) both for the probe for the emission of light (central) and for the optically coupled detection system (peripheral). Said integrated device is connected to a Fourier transform spectrometer (FTIR) with InGaAs detector, spectral range 12800-4000 cm⁻¹, resolution 2 cm⁻¹ and equipped with industrial PC plus Modbus interface for communication with the distributed control system (DCS) of the pyrolysis plant. The spectrometer used is a Bruker Matrix-F.

Said hollow duct also includes a temperature transducer for measuring the temperature of the substantially plastic material, which extends inside said duct for about ⅓ of the internal diameter of said duct, and a pressure transducer. The outlet of said hollow conduit is connected to an orifice which has the purpose of ensuring the maintenance of the required pressure in said hollow conduit.

The extruder is fed into the hopper by three gravimetric feeders.

During the tests carried out to create the calibration curves, and for the examples where the compounds are fed in batches, the melt of substantially plastic material is passed through a die, then a water and granulate bath. In continuous pyrolysis tests, on the other hand, the melt of substantially plastic material is sent to the pyrolysis reactor.

The speed of the extruder screw was kept high enough to ensure that the extruder hopper was kept empty.

### Tests to Create the Calibration Curves

For each of the sample materials, tests were carried out at 4 different temperatures (170-190-210-230° C., an interval therefore reduced compared to that used in the spectra shown in FIG. 6) and 3 different flow rates (corresponding to an average speed in the duct, calculated as a volumetric flow rate on the area of the hollow duct, equal to 1, 1.5 and 2 mm/s).

The pressure measured in the hollow duct, near the point of measurement of the spectra was variable according to the tests and the sample materials, but always between 5 and 200 bara.

10 spectra were also acquired for each condition, for a total of 120 spectra for each sample material.

### Determination of the “Cx” Calibration Curves

The calibration curves were determined according to the “rolling” mode described above, selecting 7 sample materials for verification and 51−7=44 sample materials as calibration samples for multivariate regression. The second round was performed by selecting 7 other sample materials (different from the first) for verification and the remaining ones as calibration samples for multivariate regression. We proceeded in the same way for a total of 6 laps. We proceeded in the same way also for the seventh and last round, except that the sample materials used for the verification were the remaining 51−7*6=9 not yet used in verification, and the remaining 42 as calibration samples for the multivariate regression.

The implicit mode was used, in which therefore the process variables such as temperature, pressure and speed act implicitly in the model, and not as additional parameters.

A multivariate regression of type PLS (partial least squares) was applied.

The final calibration curve uses the values of the loads (“loadings”) which is given by the average of the loads identified according to the rotation mode described above.

### Choice of Number of Components

The number of PLS components to be used in the model was evaluated.

A reduced number of components reduces the accuracy of the model, while an excessive number of components poses the risk of overfitting, reducing the extrapolative and interpolative capacity of the model.

FIG. 3 shows the result, which shows that the mean square error in prediction drops to very low values already using the first 5 components (NC=5).

Considering that for NC=11 we get very close to the minimum of the prediction error, it was chosen to use the calibration curve including the first 11 components (NC=11).

### Accuracy of Prediction of “Cx” Calibration Curves

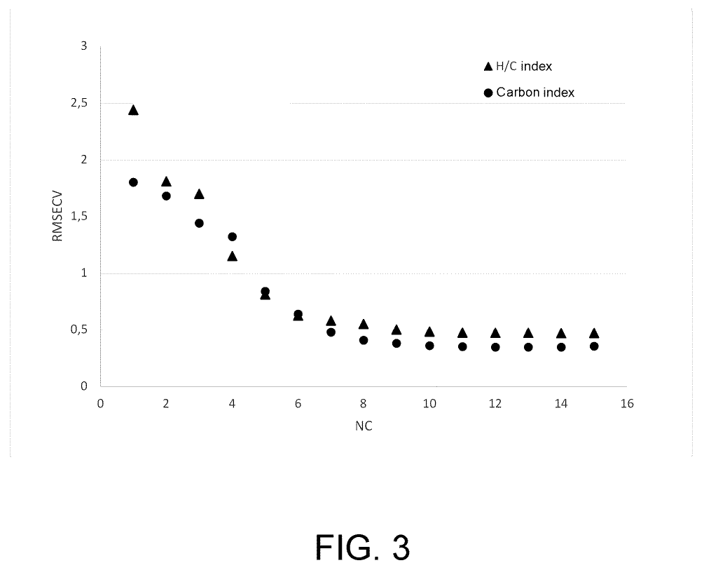

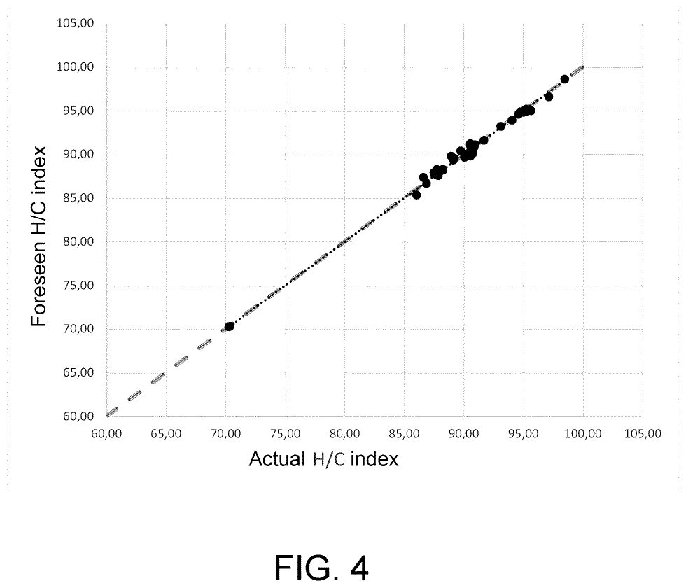

FIGS. 4 and 5 respectively show the H/C index and the carbon index calculated with said calibration curve, compared with the “true” H/C value, i.e. determined on the basis of the primary analysis. The predictive capacity of the method disclosed in the present invention and the remarkable accuracy of said prediction are clearly evident from these Figures.

### Pyrolysis Apparatus Used for the Pyrolysis Examples (“Apparatus 1”)

The pyrolysis apparatus used for the Pyrolysis Examples of the present invention consists of:
a thermostatted reactor, equipped with a flange for loading materials, a dip tube for the inerting gas (nitrogen) inlet, a nozzle for connection to a possible extruder for the entry of substantially plastic material, a nozzle for the exit of vapours and a nozzle for each of the thermocouples for measuring the temperature and measurement pressure, plus two nozzles for level measurement;
a stirring system of said reactor, equipped with an anchor stirrer, low rotation speed (tip velocity approx. 0.1 m/s) and breakwater
a flowmeter equipped with a fine adjustment valve for regulating the inerting gas flow rate into the reactor;
a pressure transducer located on the head of the reactor, plus a local pressure gauge, which read the pressure of the gases inside the reactor;
three thermocouples for measuring the actual temperature located in the lower part of the reactor;
a reactor temperature regulation system that reads the temperature value of one of the three thermocouples and acts in feedback on the thermostating system, whose control parameters have been suitably calibrated to ensure high thermal stability (lower temperature fluctuations at 5° C.);
a level indicator, through the use of a differential pressure sensor that reads the hydrostatic head in the reactor (pressure difference between the head and bottom of the reactor);
a condenser for the condensation of the vapours leaving the reactor, kept at −10° C. by means of a refrigerant fluid made to flow by a refrigeration unit at a controlled temperature;
a valve for regulating the flow of gas leaving the reactor interposed between said reactor and said condenser;
a system for regulating the pressure of the reactor which reads the pressure value of said pressure transducer and acts in feedback on said regulating valve, so as to ensure a high pressure stability (pressure oscillations lower than 50 mBar)
an expandable flask hermetically connected to the upper outlet of said condenser designed to collect the gaseous fraction that is not condensed;
a receiving container hermetically connected to the lower output of said condenser designed to collect the condensed fraction and therefore in the liquid state, with the vents connected to said upper output of the condenser;
a valve for the interception of incoming nitrogen;
a valve for the interception of the liquid product leaving the condenser, before the hermetic connection with the receiving container;
a valve for the interception of the gaseous product leaving the condenser, before the hermetic connection with the expandable flask;
an apparatus for feeding the substantially plastic material at least partially in the molten state to the pyrolysis reactor, comprising the system for measuring and analysing the spectra on the plastic material described above;
a gravimetric feeder for dosing the granulated polymeric mixture in the hopper of said twin-screw extruder for dosing the granulated polymeric mixture in the reactor.

### Examples of Preparation of the Granulated Polymer Blend

The compounds (PAT1, PAT2, PAT3, PAT4, PAT5) were prepared as per the table of compositions provided. For example, the “PAT1” compound was prepared, comprising the following materials:
42 parts of low density polyethylene (LDPE) type Riblene® FC20 manufactured by Versalis;
24 parts of linear low density polyethylene (LLDPE) type Flexirene® CL10 manufactured by Versalis;
4 parts of high density polyethylene (HDPE) type Eraclene® BC82 produced by Versalis
30 parts of polypropylene (PP) type ISPLEN® PP040 produced by Repsol.

In a Coperion ZSK 26 twin-screw extruder, the compounds thus prepared were melted at 250° C., mixed using mixing elements present in the extruder screws and passed through a die. The overall residence time in the extruder was less than one minute. The mixture of polymers thus obtained was then cooled in a liquid bath and granulated into granules with a diameter and length of about 3 mm. In this way the compounds of the PAT1, PAT2, PAT3, PAT4 and PAT5 granulated polymers were produced.

### Examples of Pyrolysis from 1 to 8 (Comparative and According to the Invention)

Examples 1 to 8 were made with the granulated polymers and the pressures indicated in the following table:

| | Example Number | | | | | | | | |
|---|---|---|---|---|---|---|---|---|---|
| | [#] | 1 | 2 | 3 | 4 | 5 | 6 | 7 | 8 |
| According to | | COMPAR- | INVEN- | COMPAR- | INVEN- | COMPAR- | INVEN- | INVEN- | COMPAR- |
| invention or | | ATIVE | TION | ATIVE | TION | ATIVE | TION | TION | ATIVE |
| comparative | | | | | | | | | |
| Polymers | [—] | PAT1 | PAT1 | PAT2 | PAT2 | PAT3 | PAT3 | PAT4 | PAT4 |
| compound | | | | | | | | | |
| Pressure | [symbol] bara] | Atm. | 5 | Atm. | 5 | Atm. | 5 | Atm. | 5 |

[symbol]

indicates data missing or illegible when filed

In the reactor of “Apparatus 1” described above, the mixture of granulated polymers indicated was loaded at room temperature until ⅓ of the geometric volume of the reactor was reached. The nozzle for the possible entry of the molten polymer from the extruder has not been used and has been plugged.

The valve for regulating the flow of gas leaving the reactor has been manually set to full open.

Nitrogen was then injected from below through the dip tube, fully opening the fine adjustment valve of the flowmeter.

The gases contained in the reactor were then removed for a time equal to 24 hours, in order to ensure the elimination of oxygen.

The valves on the outlet of the gaseous and liquid products from the condenser were then closed. Immediately afterwards, the nitrogen supply was interrupted. We then proceeded to connect the expandable flask for collecting the gases produced and the receiving container for collecting the liquids produced.

The valves at the outlet of the gaseous and liquid products from the condenser were then reopened.

The valve for regulating the flow of gas leaving the reactor has been set in automatic regulation to the value chosen for the test (in this example, 0 barg=1 bara).

Nitrogen was then injected from below through the dip tube, but setting a low flow rate, selected so that the quantity of nitrogen collected in the expandable flask before its replacement does not exceed 30% of the maximum volume of the flask.

The modality described above for the realization of examples 1-8 will be defined hereinafter as “Set-up A1”. The reactor thermoregulation system was turned on, setting the following programme:
1. first heating ramp of 4 degrees per minute, until reaching 380° C.;
2. maintaining the temperature of 380° C. for 3 hours;
3. second heating ramp of 2 degrees per minute, until reaching 430° C.;
4. maintaining the temperature of 430° C. for 3 hours;
5. third heating ramp of 2 degrees per minute, until reaching 480° C.;
6. maintaining the temperature of 480° C. for 3 hours;
7. switching off the heating.

The temperature vs time profile described above will hereinafter be referred to as the “T1 Profile”.

### Cleaning

After 12 hours from the end of the program, it was verified that the reactor temperature was below 60° C. and proceeded to interrupt the nitrogen supply, close the valves for the interception of the liquid and gaseous product leaving the condenser and open the reactor flange.

The inside of the reactor was carefully cleaned in order to drop all the dust and deposits that may have formed on the parts which were in contact with the liquid and gas produced inside it.

### Analyses

We then proceeded to completely remove all the solid material from the bottom of the reactor and to weigh it, and to detach the container for receiving the liquids and the expandable flask.

The liquid contained in the liquid receiving container was weighed and then subjected to ultracentrifugation (Thermo Scientific ultracentrifuge model Sorvall Evolution RC) at 25000 RPM for 45 minutes.

The fraction left on the bottom after ultracentrifugation (hereinafter referred to as wax fraction) and the supernatant (later referred to as oil fraction) were then separated and weighed.

The percentage of oil was calculated by dividing the weight of the oil fraction obtained by the weight of the material initially fed into the reactor.

The wax percentage was calculated by dividing the weight of the wax fraction obtained by the weight of the material initially fed into the reactor.

The percentage of carbon residue (char) was calculated by dividing the weight of the solid fraction obtained by the weight of the material initially fed into the reactor.

The mass of gas produced was calculated as the difference between the weight of the material initially fed into the reactor and the sum of the weights of the wax fraction, carbon residue (char) and oil. The gas fraction produced was calculated by dividing the mass of the gaseous fraction thus calculated by the weight of the material initially fed into the reactor.

The fractions obtained were analysed with the techniques described above. The chemical compounds identified were about 130.

For each of these chemical compounds the number of atoms of each element and, from the atomic mass of each atom, the mass fraction of the atoms was calculated.

“C5-C12 yield” means the sum of the mass of chemical compounds having from 5 to 12 carbon atoms (extremes included) in the evaporated pyrolysis product with respect to the total mass fed. Similarly, by “C21+ yield” we mean the sum of the mass of the chemical compounds having at least 21 carbon atoms in the evaporated pyrolysis product with respect to the total mass fed.

By “C5-C12 fraction” we mean the sum of the mass of chemical compounds having from 5 to 12 carbon atoms (extremes included) in a product with respect to the total mass of the same. Similarly, by “C21+ fraction” we mean the sum of the mass of the chemical compounds having at least 21 carbon atoms in a product with respect to the total mass of the same.

By evaporated pyrolysis product we therefore mean the sum of the gas fraction, oil fraction and wax fraction, but not residual solid (char).

For this purpose, the gas chromatographic analysis was carried out separately on each of the gas, oil and wax fractions, then calculating the yield of a given compound “C” as follows:

$${x}_{C , VAP} = {f}_{GAS} \cdot {x}_{C , GAS} + {f}_{OIL} \cdot {x}_{C , OIL} + {f}_{WAX} \cdot {x}_{C , WAX}$$

where:
x_{C,<FRAC}> is the mass fraction of compound C in the product fraction <FRAC> indicated (<FRAC> is GAS=gaseous fraction, OIL=oil fraction or WAX=wax fraction). In the case of the GAS fraction, the nitrogen fraction present was previously eliminated from the calculation (this being fed as an inert gas and not a pyrolysis product). The chromatogram of the GAS fraction gives the parts by volume which have been converted into parts by weight assuming that the parts by volume correspond to the parts in moles and then calculating the parts by weight knowing k the molecular weights of the compounds.
f_{OIL} e f_{WAX} are the mass fractions of the oil and wax fractions respectively, obtained by dividing the weight of the collected material by the weight of the material fed into pyrolysis. The weight of the gas produced, on the other hand, was calculated by the difference between the weight of the material fed into pyrolysis and the sum of the weights of the materials of the oil, wax and solid residue (char) fractions:

$${M}_{GAS} = {M}_{FEED} - ( {M}_{CHAR} + {M}_{OIL} + {M}_{WAX} )$$

f_{GA} instead was calculated from the ratio between M_{GAS} and M_{FEED}.

The following table summarizes the pyrolysis conditions for Examples and Comparative Examples 1 to 8 of the patent:

| | Sample number | | | | | | | | |
|---|---|---|---|---|---|---|---|---|---|
| | [#] | 1 | 2 | 3 | 4 | 5 | 6 | 7 | 8 |
| Comparative/ | [—] | Comp. | Inv. | Comp. | Inv. | Comp. | Inv. | Inv. | Comp. |
| Invention | | | | | | | | | |
| Arrangement | [#] | A1 | A1 | A1 | A1 | A1 | A1 | A1 | A1 |
| Temp. | [#] | T1 | T1 | T1 | T1 | T1 | T1 | T1 | T1 |
| Profile | | | | | | | | | |
| Polymers | [—] | PAT1 | PAT1 | PAT2 | PAT2 | PAT3 | PAT3 | PAT4 | PAT4 |
| compound | | | | | | | | | |
| Pressure | [bara] | Atm. | 5 | Atm. | 5 | Atm. | 5 | Atm. | 5 |

### Pyrolysis Examples from 9 to 11

Examples 9, 10 and 11 were carried out under the same set-up and thermal profile conditions as in Examples 1-8 (set-up A1 and temperature profile T1). The pressure was set at 3 bara. In fact, the goal was to evaluate the repeatability and therefore the reliability of the results obtained and, as is the practice often followed in the design of experiments (DOE, design of experiments), it was decided to repeat the central point 3 times.

The compound used for all three Examples was PAT5, which as previously reported has a composition which is the average of the compounds PAT1, PAT2, PAT3 and PAT4. The blend was extruded forming a granulated polymer as per Examples 1 to 8.

### Pyrolysis Examples from 12 to 13 (Comparative and According to the Invention)

The Examples according to the invention and Comparative Examples 1 and 2 (PAT1 polymer blend, pyrolysis pressure 1 and 5 bara) were repeated, but using a different thermal profile now described.

The following programme on the was set reactor thermoregulation system:
1. heating ramp of 4 degrees per minute, until reaching 430° C.;
2. maintaining the temperature of 430° C. for 6 hours;
3. switching off the heating.

The difference with respect to the previous Examples is therefore that the duration of the pyrolysis treatment was reduced from 9 to 6 hours (plus ramp time), and the temperature of the intermediate stage at 430° C. was chosen as the treatment temperature.

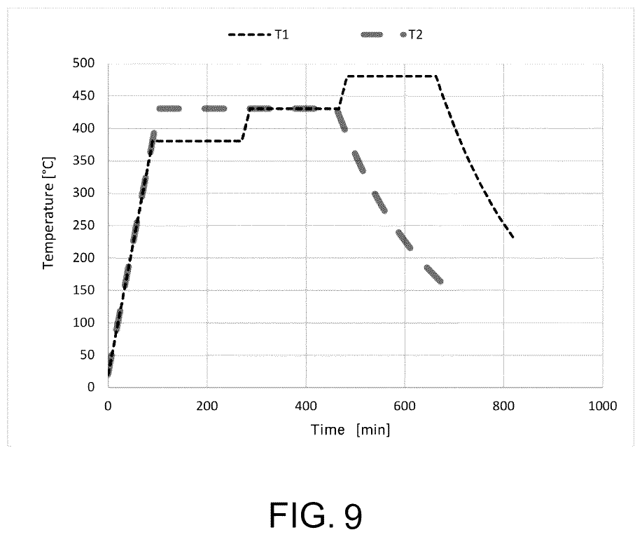

The temperature profile thus modified will later be referred to as “Profile T2”. The graph in FIG. 9 illustrates the difference. Examples 12 and 13 were carried out using the PAT1 polymer blend. The set pressure was atmospheric for example 12 and 5 bar for example 13.

### Example 14

This example relates to the pyrolysis plant managed in semi-continuous mode, and according to the “A2” set-up better specified below:
the nozzle of “Apparatus 1” for the possible entry of polymer from the extruder was opened and connected to the apparatus for feeding the substantially plastic material at least partially in the molten state to the pyrolysis reactor described above. The PAT2 granulated polymer blend was not initially loaded into the reactor, as was done in Examples 1-13 previously illustrated. The PAT2 granulated polymer blend was instead fed into the hopper of the gravimetric feeder that feeds the extruder. The speed of the extruder screw was adjusted to ensure that the extruder hopper remained empty (“empty screw”). The flow rate of the gravimetric feeder was adjusted so that the reactor level, maintained at 430° C., was equal to 40% of the total volume of the reactor. The volumetric flow rate of the polymer exiting the extruder was calculated by dividing the mass flow rate set on the dispenser by the density of the melted polymer. Before starting the feeding of the substantially plastic material, the atmosphere of the pyrolysis reactor was inserted by the entry of nitrogen as an inert gas, as done in the previous examples. The valve for regulating the flow of gas leaving the reactor has been set in automatic regulation to the value chosen for the test, as already done in the previous Examples. A time equal to 6 hours was waited to stabilise the reaction and the adjustments, during which the nitrogen inlet was closed. Once the stabilisation time was over, the residence time was measured, calculated by dividing said flow rate by the density of the substantially plastic material fed in the molten state and by the volume used in the reactor (40% of the total, as mentioned), which was equal about 6.3 hours. Unlike the batch examples, it is not possible to evaluate the mass of the solid residue until the end of the test, as the examples were carried out in succession without opening the reactor between one example and the next. Therefore, the mass of non-condensed vapours was evaluated based on the volume of the expandable flask and the density of the gas calculated on the basis of the compositional analysis of the gases present in it. The mass of the residual solid in the reactor was then calculated by the difference between the mass of substantially plastic material fed and the sum of the mass of the pyrolysis oil collected and the mass of the gas thus evaluated:

$${M}_{CHAR} = {M}_{FEED} - ( {M}_{GAS} + {M}_{OIL} + {M}_{WAX} )$$

Example 14 was carried out using the “PAT2” compound as substantially plastic material, for a further 2 hours after the first hours of stabilization (performed with the same compound), at the pressure indicated in the table, and collecting the pyrolysis products as per the previous Examples.

### Results

The following tables show the C5-C12 yield, the C21+ yield and the overall quality of the product achieved (“+” if at the same time C5-C12 yield ≥30% and C21+≤3%) as a function of the H/C index, of the carbon index, and the overall index O.I. (overall index, given by the product of the H/C index by the carbon index divided by 10000):

| | Sample Number | | | | | | | | |
|---|---|---|---|---|---|---|---|---|---|
| | [#] | 1 | 2 | 3 | 4 | 5 | 6 | 7 | 8 |
| Comparative/ | [—] | Comp. | Inv. | Comp. | Inv. | Comp. | Inv. | Inv. | Comp. |
| Invention | | | | | | | | | |
| Arrangement | [#] | A1 | A1 | A1 | A1 | A1 | A1 | A1 | A1 |
| Profile | [#] | T1 | T1 | T1 | T1 | T1 | T1 | T1 | T1 |
| Temp. | | | | | | | | | |
| Polymers | [—] | PAT1 | PAT1 | PAT2 | PAT2 | PAT3 | PAT3 | PAT4 | PAT4 |
| compound | | | | | | | | | |
| Pressure | [bara] | Atm. | 5 | Atm. | 5 | Atm. | 5 | Atm. | 5 |
| H/C index | [0-100] | 100 | 100 | 95 | 95 | 83 | 83 | 83 | 83 |
| C.I. | [0-100] | 86 | 86 | 76 | 76 | 86 | 86 | 77 | 77 |
| Carbon | | | | | | | | | |
| Index | | | | | | | | | |
| Overall | [0-1] | 0.86 | 0.86 | 0.72 | 0.72 | 0.71 | 0.71 | 0.64 | 0.64 |
| index | | | | | | | | | |
| Yield | [wt. %] | 36% | 61% | 34% | 49% | 47% | 58% | 33% | 32% |
| C5-C12 | | | | | | | | | |
| Yield | [wt. %] | 3.70% | 0.24% | 22.30% | 2.08% | 3.80% | 0.72% | 2.90% | 10.10% |
| C21+ | | | | | | | | | |
| THF | [wt. %] | 0.025% | 0.08% | 0.04% | 0.11% | 0.02% | 0.07% | 0.07% | 0.06% |
| Benzoic | [wt. %] | 0.03% | 0.00% | 0.42% | 0.08% | 1.30% | 1.00% | 2.00% | 2.10% |
| acid | | | | | | | | | |
| Isobutene | [wt. %] | 0.77% | 0.32% | 0.34% | 0.20% | 0.25% | 0.22% | 0.18% | 0.37% |
| Product | [+/−] | − | + | − | + | − | + | + | − |
| quality | | | | | | | | | |

| | Example Number | | | | | | |
|---|---|---|---|---|---|---|---|
| | [#] | 9 | 10 | 11 | 12 | 13 | 14 |
| Comparative/ | [—] | Inv | Inv | Inv. | Comp. | Inv. | Inv. |
| Invention | | | | | | | |
| Arrangement | [#] | A1 | A1 | A1 | A1 | A1 | A2 |
| Temp. | [#] | T1 | T1 | T1 | T2 | T2 | T2 |
| Profile | | | | | | | |
| Polymers | [—] | PAT5 | PAT5 | PAT5 | PAT1 | PAT1 | PAT2 |
| Compound | | | | | | | |
| Pressure | [bara] | 3 | 3 | 3 | Atm. | 5 | 5 |
| H/C index | [0-100] | 90 | 90 | 90 | 100 | 100 | 95 |
| C.I. | [0-100] | 81 | 81 | 81 | 86 | 86 | 76 |
| Carbon | | | | | | | |
| Index | | | | | | | |
| Overall | [0-1] | 0.729 | 0.729 | 0.729 | 0.86 | 0.86 | 0.72 |
| index | | | | | | | |
| Yield | [wt. %] | 47% | 49% | 48% | 33% | 55% | 50% |
| C5-C12 | | | | | | | |
| Yield C21+ | [wt. %] | 2.61% | 2.72% | 2.80% | 4.10% | 0.90% | 2.50% |
| THF | [wt. %] | 0.05% | 0.05% | 0.04% | n.d. | n.d. | n.d. |
| Benzoic | [wt. %] | 1.00% | 1.05% | 0.90% | n.d. | n.d. | n.d. |
| acid | | | | | | | |
| Isobutene | [wt. %] | 0.28% | 0.26% | 0.27% | n.d. | n.d. | n.d. |
| Product | [+/−] | + | + | + | − | + | + |
| Quality | | | | | | | |

### Discussion of the Results

One of the objectives of the invention is to solve the criticalities related to the pyrolysis of substantially plastic materials that vary greatly in composition, while maintaining a high quality of the pyrolysis products.

The PAT1, PAT2, PAT3 and PAT4 compounds were therefore chosen to evaluate the most suitable pyrolysis conditions when the incoming raw material undergoes important changes:
PAT1 is made up exclusively of vinyl polymers (polythene and polypropylene);
PAT2 includes a large amount of cellulose (20.2%)
PAT3 contains a large amount of polystyrene (27%)
PAT4 contains a high amount of polyethylene terephthalate (PET) (26.5%).

The experimentation carried out shows that there is no process condition which is optimal with respect to said objective of the present invention for all the compositions of incoming raw materials.

In particular, it is very clear that where there is a high H/C index and a high Carbon Index it is very advantageous to carry out pyrolysis at a higher than atmospheric pressure: in Example 2 conducted at 5 bara compared to Example 1 (comparative) conducted at atmospheric pressure the yield in C5-C12 is almost doubled, also drastically reducing the yield of the undesired fractions C21 or higher (C21+) from 3.7 to 0.24%.

Comparative Example 3 and Example 4 according to the invention show the effect of pressure when using a raw material with an H/C index similar to Examples 1 and 2 but with a reduced Carbon Index (from 86 to 76). Also in this case there is a significant advantage in operating pyrolysis at high pressure, even if the advantage is reduced (C5-C12 yield goes from 34% to 49% while C21+ yield drops from 22.3 to 2.1%).

Comparative Example 5 and Example 6 according to the invention show the effect of pressure when a raw material with carbon index substantially equal to Examples 1 is used and 2 (86 in both cases) but with a significant reduction in the H/C index (from 100 to 83). Also in this case there is a significant advantage in operating pyrolysis at high pressure, even if the advantage is reduced (C5-C12 yield goes from 47% to 58% while C21+ yield drops from 3.8% to 0.7%).

Comparative Example 7 and Example 8 according to the invention show the effect of pressure when using a raw material with carbon index substantially equal to the low value used in Examples 3 and 4 (77, very close to the value of 76 of the Examples 3 and 4) and with H/C index substantially equal to the low value used in Examples 5 and 6 (83 in both cases).

Surprisingly, in this case there was no advantage in increasing the pyrolysis pressure: the yield in C5-C12 in fact remained substantially constant (if not reduced from 33% to 32%). However, it is very important to note that the C21+ yield has risen tremendously, from 2.9% to over 10%. Therefore it is evident that while for some raw material mixtures it is advantageous to operate at high pressure, for other raw material mixtures it is advantageous not to operate at high pressure.

Furthermore, even with respect to the preferred objective of obtaining a C5-C12 yield equal to at least 30%, even more preferably at least 40%, and at the same time a C21 and higher yield (C21+) equal at most to 3%, it is evident that the use of the high pressure system makes it possible to reach this objective only in some conditions (PAT1, PAT2, PAT3) while in others (PAT4) it is convenient to keep the system at a lower pressure.

From this evidence, which the Applicant first noticed, the opportunity arises to adapt the process conditions according to the composition of the substantially plastic fed material. Since, as previously mentioned, this compound can vary during the process (especially when this is carried out in semi-continuous or continuous mode), the process of the present invention advantageously allows adjusting the pressure during the pyrolysis phase, and is therefore able to maximize the result as the raw material entering the pyrolysis varies;

preferably, the process of the invention is also capable of achieving the preferred objective of obtaining a C5-C12 yield of at least 30%, more preferably at least 40%, and at the same time a C21 and higher yield (C21+) equal to at most 3%, by varying the pyrolysis pressure.

Compared to the even more preferred mode in which the pyrolysis process is carried out at a pressure equal to at least a threshold pressure PS when the Overall Index, equal to the C.I. index multiplied by the H/C index divided by 10000) results greater than or equal to 0.7, and at a pressure lower than said threshold pressure PS when the Overall Index is less than 0.7, it should be noted that the use of this criterion has made it possible to obtain the production of a product that maximizes the C5 fraction-C12 minimizing over time the C21+ yield, in particular for values of said pressure between 2.0 and 2.9 bara.

In fact, by applying this criterion in the examples of the patent from 1 to 11, a yield in C5-C12 equal to at least 30% was obtained, and at the same time a yield in C21 and higher (C21+) equal to at most 3%.

Furthermore, the C21+ fraction in the pyrolysis oil obtained was no more than 3.5%, just as the C5-C12 fraction in the pyrolysis oil was at least 35%.

The process of the present invention proved to be very repeatable, as shown in Examples 9 to 11.

Examples 12 and 13 also show that similar results conforming to the teachings of the present invention can also be obtained by using different thermal profiles.

Surprisingly, the process of the present invention has made it possible to carry out the pyrolysis of substantially plastic material having very high quantities of polymers/materials with a low carbon content, with a carbon index even lower than 80, and with a high oxygen content, in particular a material substantially plastic with over 20% cellulose, without showing management or fouling problems, and with a high yield in C5-C12 and reduced yield in C21-and-higher fraction, both in batch mode (Example 4) and in semi-continuous (Example 14).

1. A process for the pyrolysis of substantially plastic material to obtain at least hydrocarbons that are in the e liquid phase at 25° C. comprising the following steps:
a) feeding a substantially plastic material at least in part in the molten state to a pyrolysis reactor;
b) subjecting said substantially plastic material at least in part in the molten state fed to a pyrolysis reactor to at least one analytical measurement “Ax” with in-line mode;
c) determining the value of at least one property “Px” of said substantially plastic material by means of correlation of said at least one analytical measurement “Ax” with at least one calibration curve “Cx” obtained from sample materials having a known value of the property “Px”;
d) setting at least one “Ox” parameter of the pyrolysis process based on the value of said at least one “Px” property; characterized by the fact that said at least one analytical measurement “Ax” provides for or consists in the measurement of the spectrum in reflection of the light of the substantially plastic material at least in part in the molten state,
and by the fact that said at least one property “Px” of said substantially plastic material is the H/C index and/or the carbon index, wherein the carbon index and the hydrogen/carbon ratio (H/C index) are calculated according to the following formulas:
$$H / C Index = 100 \cdot \frac{12}{2} \cdot \frac{Weight H atoms}{Weight C atoms} \\ Carbon Index = 100 \cdot \frac{Weight C atoms}{Weight ALL atoms}$$
wherein said weights of the atoms are the weight of the atoms indicated in the substantially plastic material in its entirety.

2. The process for the pyrolysis of substantially plastic material according to claim 1, in which the latency time between said at least one analytical measurement “Ax” and the setting of said at least one parameter “Ox” of the pyrolysis process is not more than 1 hour, or no more than 10 minutes, or between 1 and 60 seconds.

3. The process for the pyrolysis of substantially plastic material according to claim 1, wherein said determination of the value of at least one property “Px” of said substantially plastic material is carried out by means of a correlation of said analytical measurement “Ax” with the analytical measurement obtained by acquiring a plurality of model absorbances of plastic materials having a known value of said at least one property “Px”.

4. The process for the pyrolysis of substantially plastic material according to claim 1, wherein said at least one parameter “Ox” of the pyrolysis process is selected from the pressure of the pyrolysis reactor, the temperature of the pyrolysis reactor, the flow rate of the substantially plastic material fed to said pyrolysis reactor, the residence time of the substantially plastic material in said pyrolysis reactor and a combination of said parameters.

5. The process for the pyrolysis of substantially plastic material according to claim 4, wherein said at least one parameter “Ox” of the pyrolysis process is the pressure of the pyrolysis reactor.

6. The process for the pyrolysis of substantially plastic material according to claim 3, wherein said at least one parameter “Ox” of the pyrolysis process is determined by means of an expression which is a function of the “Overall index” (O.I.), defined as:
$$O . I . = \frac{[ C . I . ] \cdot [ H / C idx ]}{10000}$$
wherein C.I. is the carbon index and H/C idx is the H/C index.

7. The process for the pyrolysis of substantially plastic material according to claim 6, wherein said pressure is set at least a threshold pressure PS when the “Overall index” (O.I.) is greater than or equal to 0.7, and at a pressure lower than said pressure threshold when the “Overall index” (O.I.) is less than 0.7, and where preferably said threshold pressure PS is at least 1.5 bar, even more preferably between 2 and 2.9 bara, in particular 2.5 bara.

8. A method for determining the value of at least one property “Px” of a substantially plastic material which includes the following steps:
i) calculating, for each of said at least one property “Px”, a calibration curve “Cx” which, for sample materials having a known value of the property “Px”, correlates the spectrum in reflection with said property “Px”;
ii) carrying out at least one measurement “Ax” of the reflection spectrum of said substantially plastic material;
iii) determining the value of said at least one property “Px” of said substantially plastic material by means of a correlation of said at least one measurement “Ax” with said at least one calibration curve “Cx”;
and which is characterised in that said substantially plastic material, in the spectrum measurement conditions in reflection, is in the molten state for at least 60% by weight, or between 70% and 99% by weight, or between 80% and 94% by weight of the material, with respect to the overall weight of the substantially plastic material, and by the fact that said at least one property “Px” of said substantially plastic material is the H/C index and/or the carbon index, wherein the carbon index and the hydrogen/carbon ratio (H/C index) are calculated according to the following formulas:
$$H / C Index = 100 \cdot \frac{12}{2} \cdot \frac{Weight H atoms}{Weight C atoms} \\ Carbon Index = 100 \cdot \frac{Weight C atoms}{Weight ALL atoms}$$
wherein said weights of the atoms are the weight of the atoms indicated in the substantially plastic material in its entirety.

9. The method for determining the value of at least one property “Px” according to claim 8, wherein the substantially plastic material, under the conditions of measurement of spectrum in reflection, has a temperature between 14° and 300° C., or between 16° and 260° C.

10. The method for determining the value of at least one property “Px” according to claim 8, wherein the substantially plastic material, under the measuring conditions of spectrum in reflection, it has a pressure of not less than 2 bar, or between 3 and 300 bara.

11. The method for determining the value of at least one property “Px” according to claim 8, wherein at least one property “Px” is at least two.

12. The method for determining the value of at least one property “Px” according to claim 8, wherein the reflection spectrum of said substantially plastic material acquired in step ii) is in the range between 4000 and 12000 cm⁻¹, or is in the range of 4500 to 10000 cm⁻¹, or is in the range of 5000 to 9000 cm⁻¹.

13. The method for determining the value of at least one property “Px” according to claim 8, wherein the calibration curve “Cx” is obtained by applying a multivariate regression method.

14. The method for determining the value of at least one property “Px” according to claim 13, wherein said multivariate regression is chosen between multiple linear regression (MLR), regression by principal component analysis (PCR) and regression by partial least squares (PLS).

15. The method for determining the value of at least one “Px” property according to claim 14, wherein said multivariate regression is principal component analysis (PCR) regression or partial least squares (PLS) regression.

16. The method for determining the value of at least one property “Px” according to claim 15, wherein said calibration curve “Cx” is calculated on the first from 5 to 15 principal components, or on the first from 8 to 11 principal components.

17. The method for determining the value of at least one property “Px” according to claim 15, wherein the calibration curve “Cx” is obtained by carrying out the measurement in different temperature conditions of the sample material.

18. The method for determining the value of at least one property “Px” according to claim 16, wherein said calibration curve “Cx” also includes as a variable the temperature of the sample material.

19. An apparatus for determining the value of at least one property f a substantially plastic material at least in part in the molten state, wherein said at least one property “Px” of said substantially plastic material is the H/C index and/or the carbon index, wherein the carbon index and the hydrogen/carbon ratio (H/C index) are calculated according to the following formulas:
$$H / C Index = 100 \cdot \frac{12}{2} \cdot \frac{Weight H atoms}{Weight C atoms} \\ Carbon Index = 100 \cdot \frac{Weight C atoms}{Weight ALL atoms}$$
wherein said weights of the atoms are the weight of the atoms indicated in the substantially plastic material in its entirety, wherein said apparatus includes:
1) a cavity in which said substantially plastic material can slide, provided with an inlet and an outlet for said substantially plastic material at least in part in the molten state;
2) at least one probe capable of emitting light in said cavity;
3) at least one detection system optically coupled with said probe for detecting the reflected light and the determination of at least one analytical measurement “Ax”;
4) at least one sensor for reading the temperature connected to said cavity,
5) a system for determining said at least one property “Px” of said substantially plastic material at least in part in the molten state by correlating said at least one analytical measurement “Ax” with at least one calibration curve “Cx” obtained from sample materials having a known value of the “Px” property,
wherein said cavity is able to withstand a pressure of at least 50 bara, or between 80 and 300 bara.

20. The apparatus for determining the value of at least one property “Px” according to claim 19, wherein said sensor for the temperature reading is placed so that the minimum distance between the temperature reading area and the area where the reflection spectrum is measured is not more than 100 mm, or not more than 30 mm, or not more than 3 mm.

21. The apparatus for determining the value of at least one property “Px” according to claim 19, wherein said at least one detection system optically coupled with said probe is positioned so that the minimum distance between the emission zone of the probe and the detection zone of said emission is not more than 50 mm, or not more than 15 mm, or not more than 3 mm.

22. The apparatus for determining the value of at least one property “Px” according to claim 19 in which said cavity is a part of an extruder cylinder, or a part of a conveyor screw, or a connected pipe section to a pyrolysis reactor.
